(19) United States
(12) Patent Application Publication
(10) Pub. No.: US 2023/0270714 A1
(43) Pub. Date: Aug. 31, 2023

(54) SALVINORIN COMPOSITIONS

(71) Applicant: atai Life Sciences AG, Berlin (DE)

(72) Inventors: Prerak Patel, Flemington, NJ (US); Majed Fawaz, Foxboro, MA (US); Srinivas Rao, Encinitas, CA (US); Glenn Short, Scituate, MA (US); Santnu Patel, South Setauket, NY (US)

(21) Appl. No.: 18/077,844

(22) Filed: Dec. 8, 2022

Related U.S. Application Data
(60) Provisional application No. 63/287,210, filed on Dec. 8, 2021.

(51) Int. Cl. A61K 31/366; A61K 47/40; A61K 45/06; A61K 9/08; A61K 9/00

(52) U.S. Cl. CPC A61K 31/366; A61K 9/006; A61K 9/08; A61K 9/0019; A61K 45/06; A61K 47/40

(57) ABSTRACT

The disclosure provides compositions comprising Salvinorin A or a derivative of Salvinorin A for use in treating neurological diseases and conditions.

### RELATED APPLICATIONS

The application is related to and claims the benefit of priority to U.S. Provisional Pat. Application Serial No. 63/287,210, filed on Dec. 8, 2021 which is incorporated herein by reference in its entirety.

### FIELD

The disclosure relates to novel salvinorin compositions and uses thereof. Specifically, the disclosure provides pharmaceutical compositions comprising Salvinorin A or a derivative of Salvinorin A with desirable pharmacokinetic profiles (e.g., delayed onset and offset). In particular, the compositions are formulated for administration of Salvinorin A or a derivative of Salvinorin A to a subject buccally or by subcutaneous injection. Such compositions may be especially useful for the treatment of neurological diseases and conditions, including depression and treatment resistant depression.

### BACKGROUND

Salvinorin A is an active component of Salvia divinorum, a perennial herb of the Lamiaceae (mint) family, indigenous to Mexico. Preparations of Salvia divinorum have been used traditionally to produce visionary states of consciousness during spiritual healing sessions for religious purposes. When chewed or smoked, Salvia divinorum causes visual hallucinations and behavioral impairment within seconds that last only minutes.

Salvinorin A is the only known naturally-occurring, non-nitrogenous kappa opioid receptor (KOR) agonist. Unlike opiates and other KOR agonists, Salvinorin A does not induce the release of dopamine in the nucleus accumbens region of the brain that excites the brain reward system attributable to addictiveness. However, although the intrinsic properties of Salvinorin A make it an attractive possible medication, especially for neurological diseases and conditions, the compound’s very rapid onset and offset using current therapeutic compositions and modes of administration, including intravenously and via inhalation, complicate treatment. In particular, it is challenging to determine a suitable administration regimen with appropriate dosage, duration of exposure, route of administration, and frequency of administration of Salvinorin A which provides effective therapy for neurological diseases and conditions, while minimizing concomitant undesirable side effects. These side effects, which include memory loss and persistent impairment of motor skills, limit the potential use of Salvinorin A to effectively treat a variety of neurological diseases and conditions.

In additon, when administered orally, Salvinorin A fails to exhibit any psychoactive effects, even at higher doses, due its enzymatic deactivation (to its inactive metabolite, Salvinorin B) within the gastrointestinal tract. As such, a conventional oral formulation of Salvinorin A has not been realized and may be nonviable.

Therefore, a significant medical need exists for readily administrable medications of Salvinorin A or a Salvinorin A derivative to treat a variety of neurological diseases and conditions. Such medications, which maximize efficacy (e.g., avoiding gastrointestinal and / or first-pass metabolism) while effectively controlling side effects of the drug, are of particular interest. The disclosure solves this medical need by providing novel compositions comprising Salvinorin A or a Salvinorin A derivative with desirable solubility, stability, and pharmacokinetic characteristics, such as delayed onset and offset, particularly when administered via non-oral routes (i.e., a buccal / sublingual, or subcutaneous route).

### SUMMARY

In one aspect, the disclosure provides a pharmaceutical composition comprising Salvinorin A or derivative of Salvinorin A, wherein following administration (e.g., buccally or by subcutaneous injection) to a human subject, the Salvinorin A or Salvinorin A derivative human blood plasma Tₘₐₓ is between from about 10 minutes to about 240 minutes.

In one aspect, the disclosure provides a buccal pharmaceutical composition comprising Salvinorin A or derivative of Salvinorin A, and a buccal film matrix comprising a solvent, a co-solvent, a surfactant, and a polymer. In some embodiments the film matrix further comprises a permeability enhancer and/or an oil. In some embodiments, the polymer comprises povidone, copovidone, or hydroxypropyl cellulose or combinations thereof. In some embodiments the solvent comprises N-methylpyrrolidone, methanol, ethanol, methyethylketone, or acetone or combinations thereof. In some further embodiments the solvent comprises N-methylpyrrolidone, polyethylene glycol or methanol or combinations thereof. In some embodiments, the surfactant comprises Lauroyl Polyoxyl-32 glycerides or D-α-tocopheryl polyethylene glycol succinate, or combinations thereof. In some embodiments, the co-solvent comprises propylene glycol or benzyl alcohol or combinations thereof.

In one aspect, the disclosure provides an injectable subcutaneous pharmaceutical composition comprising Salvinorin A or derivative of Salvinorin A, and a vehicle comprising a solvent, surfactant, and/or a co-solvent. In some embodiments the solvent comprises a polar aprotic solvent or an oil. In some further embodiments, the solvent comprises a pyrrolidone or an oil. In some further embodiments, the pyrrolidone comprises N-methyl pyrrolidone (NMP). In some embodiments the co-solvent comprises a polyethylene glycol. In some further embodiments the polyethylene glycol comprises PEG300 or PEG400. In some further embodiments, the surfactant comprises a fatty acid ester. In yet further embodiments the fatty acid ester comprises caprylocaproyl macrogoglycerides. In some embodiments, the vehicle further comprises a complexing agent comprising a cyclodextrin. In some further embodiments, the cyclodextrin comprises 2-hydroxypropyl-beta-cyclodextrin. In some embodiments the vehicle further comprises a stabilizer, surfactant, precipitation inhibitor, preservative, and/or an antioxidant.

In one aspect, the disclosure provides a pharmaceutical composition comprising Salvinorin A or derivative of Salvinorin A, wherein following administration (e.g., buccally or by subcutaneous injection) to a human subject, the Salvinorin A or Salvinorin A derivative human blood plasma Tₘₐₓ is between from about 10 minutes to about 240 minutes, about 10 minutes to about 180 minutes, about 10 minutes to about 120 minutes, or about 10 minutes to about 60 minutes, and after Tₘₐₓ is achieved, the concentration of Salvinorin A or derivative of Salvinorin A is maintained at about 50% or more of Cₘₐₓ for between about 10 minutes to about 90 minutes (e.g., about 10, about 15, about 20, about 25, about 30, about 35, about 40, about 45, about 50, about 55, about 60, about 65, about 70, about 75, about 80, about 85, or about 90 minutes).

In one aspect, the disclosure provides a pharmaceutical composition comprising Salvinorin A or derivative of Salvinorin A, wherein following administration to a human subject, the Salvinorin A or Salvinorin A derivative human blood plasma Tₘₐₓ is between from about 10 minutes to about 240 minutes, and after Tₘₐₓ is achieved, the concentration of Salvinorin A or derivative of Salvinorin A is maintained at about 80% or more of Cₘₐₓ for between about 10 minutes to about 45 minutes (e.g. about 10, about 15, about 20, about 25, about 30, about 35, about 40 minutes or about 45 minutes).

In one aspect, the disclosure provides a pharmaceutical composition comprising Salvinorin A or derivative of Salvinorin A, wherein following administration, therapeutically effective blood levels of Salvinorin A or Salvinorin A derivative are maintained for at least about 20 minutes or longer (e.g., about 20, about 25, about 30, about 35, about 40, about 45, about 50, about 55, about 60, about 65, about 70, about 85, about 90, about 95, about 100, about 105, about 110, about 115, about 120, about 125, about 130, about 135, about 140, about 145, about 150, about 155, about 160, about 165, about 170, about 185, about 190, about 195, about 200, about 205, about 210, about 215, about 220, about 225, about 230, about 235, or about 240 minutes). In some embodiments of this aspect, therapeutically effective blood levels of Salvinorin A or Salvinorin A derivative are maintained for a time within a range of about 20 minutes to about 240 minutes, about 20 minutes to about 180 minutes, about 20 minutes to about 150 minutes, about 20 minutes to about 120 minutes, about 20 minutes to about 100 minutes, about 20 minutes to about 80 minutes, about 20 minutes to about 60 minutes, or about 20 minutes to about 40 minutes. In some further embodiments, therapeutically effective blood levels of Salvinorin A or Salvinorin A derivative are maintained for a time within a range of about 30 or 40 minutes to about 240 minutes, about 30 or 40 minutes to about 180 minutes, about 30 or 40 minutes to about 150 minutes, about 30 or 40 minutes to about 120 minutes, about 30 or 40 minutes to about 100 minutes, about 30 or 40 minutes to about 80 minutes, about 30 or 40 minutes to about 60 minutes, or about 30 minutes to about 40 minutes. In one embodiment of this aspect, blood levels of Salvinorin A or Salvinorin A derivative are maintained at a concentration below a concentration that is likely to cause undesirable side effects, such as memory loss.

In one aspect, the disclosure provides a pharmaceutical composition comprising Salvinorin A or derivative of Salvinorin A, wherein following administration (e.g. buccally or by subcutaneous injection to a human subject), at least 50% (e.g. about 50%, about 55%, about 60%, about 65%, about 70%, or about 75%) of the drug is released from the composition between about 10 minutes to about 60 minutes, about 10 minutes to about 50 minutes, about 10 minutes to about 40 minutes, or about 10 minutes to about 30 minutes.

In one embodiment of any of the previous aspects, the human blood plasma T½ is about 15 minutes to about 240 minutes, about 15 minutes to about 220 minutes, about 15 minutes to about 200 minutes, about 15 minutes to about 180 minutes, about 15 minutes to about 150 minutes, about 15 minutes to about 120 minutes, about 15 minutes to about 100 minutes, or about 15 minutes to about 90 minutes. In some further embodiments, T½ is about 20 minutes, about 30 minutes, about 40 minutes, about 50 minutes, about 60 minutes, about 70 minutes, about 80 minutes, or about 90 minutes.

In some embodiments of any of the previous aspects, the formulation comprises Salvinorin A.

In some embodiment of the previous aspects, the formulation is administered buccally. In a particular embodiment, the drug is absorbed across the buccal mucosa.

In some embodiments of any of the previous aspects, the formulation is administered by subcutaneous injection.

In embodiments of any of the previous aspects, Tₘₐₓ is 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 210, 220, 230, or 240 minutes.

In one aspect, the disclosure provides a method of treating a neurological disease or condition, without inducing significant side effects, such as memory loss, comprising buccally administering to a human subject an effective amount of Salvinorin A or a derivative of Salvinorin A. In one embodiment of this aspect, the drug is absorbed across the buccal mucosa. In one embodiment of this aspect, the method comprises treatment of depression, including treatment resistant depression.

In one aspect, the disclosure provides a method of treating a neurological disease or condition, without inducing significant side effects, such as memory loss, comprising administering by subcutaneous injection to a human subject an effective amount of Salvinorin A or a derivative of Salvinorin A. In one embodiment of this aspect, the method comprises treatment of depression, including treatment resistant depression.

### BRIEF DESCRIPTION OF THE DRAWINGS

FIG. 1. depicts the concentration of Salvinorin A in human blood plasma over time following buccal administration or by subcutaneous injection of a pharmaceutical composition comprising Salvinorin A in accordance with the example aspects and embodiments of the disclosure.

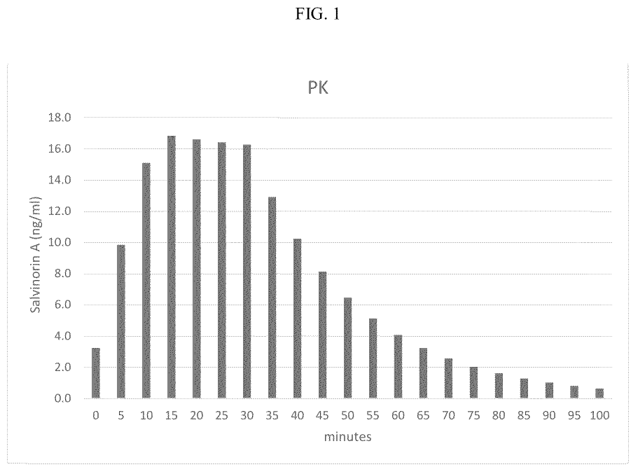

FIG. 2. depicts the percentage release of Salvinorin A over time following the buccal administration or by subcutaneous injection of a pharmaceutical composition comprising Salvinorin A in accordance with the example aspects and embodiments of the disclosure.

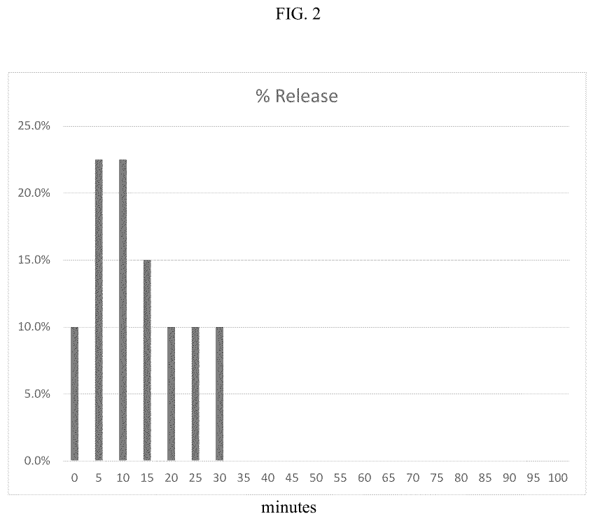

FIG. 3. depicts the stability of Salvinorin A over time in three solution formulations in accordance with the example aspects and embodiments of the disclosure.

FIG. 4. depicts the solubility of Salvinorin A (API) or Salvinorin A film compositions in 10 mM PBS, pH 7.0 in accordance with the example aspects and embodiments of the disclosure.

FIG. 5. depicts the dissolution profile and percent drug release of Salvinorin A film formulations in accordance with the example aspects and embodiments of the disclosure.

### DETAILED DESCRIPTION OF THE INVENTION

Drugs can be absorbed through mucosal surfaces, such as those in the oral cavity. Drug delivery via mucosal surfaces can be efficient because they lack the stratum corneum of the epidermis, a major barrier to absorption across the skin. Mucosal surfaces are also typically rich in blood supply, which can rapidly transport drugs systemically while avoiding significant degradation by first-pass hepatic metabolism.

Oral transmucosal absorption is generally rapid because of the rich vascular supply to the mucosa and the lack of a stratum corneum in the epidermis. Such drug transport typically provides a rapid rise in blood concentrations, and similarly avoids the enterohepatic circulation and immediate destruction by gastric acid or partial first-pass effects of gut wall and hepatic metabolism. Drugs typically need to have prolonged exposure to an oral mucosal surface for significant drug absorption to occur. Factors affecting drug delivery include taste, which can affect contact time, and drug ionization. Drug absorption is generally greater from the buccal or oral mucosa than from the tongue and gingiva. One limitation commonly associated with buccal drug delivery is low flux, which often results in low drug bioavailability. Low flux may be somewhat offset by using buccal penetration enhancers, as are known in the art, to increase the flux of drugs through the mucosa. Drugs may also be given transmucosally by vaginal or rectal delivery means.

When administered transmucosally, for example by a buccal route, drug absorption can be delayed or prolonged, or uptake may be almost as rapid as if an intravenous bolus were administered. The disclosure is based, in one aspect, on the development of certain buccal compositions of Salvinorin A or a derivative of Salvinorin A which provide a delayed onset and offset of the drug following administration to the human subject in need thereof compared to the onset and offset of the drug following administration intravenously or via inhalation.

From a pharmacokinetic point of view, parenteral routes are often considered near-ideal ways of administration due to the high bioavailability and rapid onset of action usually obtained. In the case of intravenous administration, the entire dose reaches the systemic circulation and an immediate physiological response can be achieved. In contrast, intramuscular and subcutaneous administrations involve an absorption process from the injection site, which leads to a delayed response, since drug molecules have to diffuse in the interstitial space in order to reach the capillaries (i.e., to be absorbed). This absorption process can be influenced by various factors, either physicochemical (such as molecular size, electrostatic charge, and hydrophilicity) or physiological (such as arising from the interaction of the administered drug with endogenous compounds, blood, and lymph flows and/or the influence of tissue hydration). The disclosure is based, in one aspect, on the development of certain compositions of Salvinorin A or a derivative of Salvinorin A for subcutaneous administration which provide a delayed onset and offset of the drug following administration to the human subject in need thereof compared to the onset and offset of the drug following administration intravenously or via inhalation (e.g., smoke).

Administration by subcutaneous injection has certain advantages over intravenous and intramuscular injection. For example, skilled personnel are not required when administering a drug subcutaneously, in contrast to intravenous and intramuscular administration. Furthermore, injecting subcutaneously is less painful than injecting into a vein or muscle. Also, the risk of infection is lower when administering a drug subcutaneously compared to an intravenous injection, and, if an infection occurs following subcutaneous administration, the infection is generally limited to a local infection rather than a systemic infection with an intravenous injection. There are also a wider range of alternative injection sites for a subcutaneous injection than there are for an intramuscular injection, which is especially important for patients requiring multiple doses.

As pain from injection can lead to non-compliance, short and thin needles, conveniently lubricated and with sharp tips, are generally used to minimize pain when administering a subcutaneous injection. Large subcutaneous injection volumes can also be associated with pain. Therefore, the maximum volume generally accepted when injecting subcutaneously into the thigh is about 1.5 ml, although volumes of up to 4 ml (e.g. up to about 3 ml) are well tolerated when injected subcutaneously in the abdomen. Ideally, the subcutaneous injection should be formulated as an isotonic solution (e.g. with osmolality of about 300 mOsm/kg) to prevent pain, although a hypertonic solution to about 600 mOs/kg may be used. The viscosity of the injected solution should also be carefully selected since a very low viscosity solution may be associated with an increased pain sensation. A pH close to physiological pH is also recommended to minimize pain, irritation, and tissue damage. Buffers, such as citrate or phosphate buffers, are frequently added to parenteral formulations, including formulations for subcutaneous injection, to optimize solubility and stability by adjusting the pH. However, their strength should be kept low to avoid pain upon injection. For a review of factors influencing pain at the injection site see: Iris Usach et al. in Adv Ther. (2019) 36:2986-2996.

The unique PK profile obtained for compositions in accordance with the disclosure offers advantages in the treatment of neurological diseases or conditions over traditional routes of administration, such as via inhalation or intravenous injection which exhibit a very rapid onset and offset, or oral dosage forms which can lack efficacy (e.g., via conversion to inactive forms of active agent).

Thus, the disclosure relates to pharmaceutical compositions comprising Salvinorin A or a derivative of Salvinorin A, together with uses thereof, particularly for the treatment of a neurological disease or condition. Specifically, the disclosure provides pharmaceutical compositions comprising Salvinorin A or a derivative of Salvinorin A which exhibit favorable pharmacokinetic characteristics when administered to a subject (e.g.,, human patient). As described herein the pharmaceutical compositions or formulations comprising Salvinorin A or a derivated thereof provide any one or more of desirable solubility, stability, and pharmacokinetic characteristics, such as delayed onset and offset, particularly when administered via non-oral routes to a human patient. In one embodiment, the desired delayed onset and offset is achieved following buccal administration. In another embodiment, the desired delayed onset and offset is achieved following subcutaneous injection to a human subject. In embodiments, the pharmaceutical compositions can increase solubility and/or stability of Salvinorin A or derivative thereof, relative to other compositions that comprise Salvinorin A (i.e., other formulations administered via injectable, oral/buccal, transmucosal, inhaled, etc., routes).

### DEFINITIONS

As used herein, the following terms are defined with the following meanings, unless explicitly stated otherwise.

Salvinorin A is a selective kappa-opioid-receptor (KOR) agonist described by Roth BL et al., in Proc. Natl. Acad. Sci. USA 2002; 99:11934-11939. DOI: 10.1073/pnas.182234399. [PubMed: 12192085]. As used herein, the term “Salvinorin A” includes substantially pure Salvinorin A (e.g., at least 95% pure) and crystalline Salvinorin A, including crystalline Salvinorin A hydrate and all polymorphic forms thereof.

The term “Salvinorin A derivative” means a compound structurally related to Salvinorin A, including an analog of Salvinorin A, which exhibits KOR agonist activity. Examples of Salvinorin A derivatives, which are incorporated by reference herein, include compounds described in US2006/0052439, US2007/0213394, US2010/0324131, WO2005/089745, WO2010/075045, US2012/0010219, WO2020/131689A1, WO2006/031782, and WO2006/012643.

The term “about” when used before a numerical designation, e.g., pH, temperature, amount, or concentration, indicates an approximation which may vary by amounts that do not have any significant effect on the resulting structure, stability, activity, or result-effective variable or parameter. In some embodiments the term about can allow for various relative amounts of the particular element or variable such as, for example, of up to (+) or (–) 5% or even up to (+) or (–) 10%.

The singular form “a”, “an” and “the” include plural references unless the context clearly dictates otherwise. For example, the term “a pharmaceutically acceptable carrier” may include a plurality of pharmaceutically acceptable carriers, including mixtures thereof.

The term “and/or” is intended to mean either or both of two components of the invention.

The term “subject,” “individual” and “patient” are used interchangeably herein, and refers to a mammal, and is preferably a human.

The term “device,” as used herein, refers to an apparatus or system capable of delivering a drug to a patient in need thereof.

The term “in need of treatment” and the term “in need thereof” when referring to treatment are used interchangeably and refer to a judgment made by a caregiver (e.g. physician, nurse, nurse practitioner) that a patient will benefit from treatment. In embodiments, the patient can present with one or more clinical symptoms and/or one or more risk factors associated with a disease or condition to be treated.

The terms “treat” and “treatment” refer herein to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition. “Treatment” can also mean prolonging survival as compared to expected survival if not receiving treatment. Those in need of treatment include those already with the disease or condition as well as those prone to having the disease or condition or those in which the disease or condition is to be prevented.

The term “buccal delivery” or “buccal administration” refers to a route of administration in which the pharmaceutical dosage form is applied between the patient’s cheek and gum (i.e. the buccal cavity).

The term “pharmaceutically acceptable,” as used herein, refers to a component of a pharmaceutical composition that is compatible with the other ingredients of the formulation and not overly deleterious to the recipient thereof.

The term “carrier” refers to a diluent, adjuvant, excipient, or vehicle with which the therapeutic is administered and includes, but is not limited to, such liquids and powders that are hydrophilic substances, hydrophobic substances and substances that possess both hydrophilic and hydrophobic properties such as emulsifiers.

The term “effective amount” or “therapeutically effective amount” as used herein, refers to the amount of active agent that elicits the biological or medicinal response in a tissue, system, or individual that is being sought by a researcher, healthcare provider or individual.

The term “neurological disease or condition” as used herein, means a disease or condition selected from: a neuropsychiatric disorder, such as depression (including severe depression such as treatment-resistant depression), anxiety, bipolar disorder, post-traumatic stress disorder, abnormalities of mood or emotion, including the above conditions, dysthymia, schizoaffective disorder, schizophrenia and other psychotic disorders, panic disorder, traumatic stress disorders, phobic disorders, and personality disorders with abnormal mood, such as borderline personality disorder, schizoid and schizotypal disorders and suicide ideation, or rumination/unproductive repetitive thoughts negatively impacting one’s behavior/mood/ability to focus; addiction (including substance use disorder such as addiction to nicotine, alcohol, cocaine, opioids, amphetamine, methamphetamine, heroin, morphine, phencyclidine, 3,4-methylenedioxy-methamphetamine, as well as other addictive substances); addictive behavior (including eating, gambling, sex, pornography, videogames, work, exercise, spiritual obsession, self-harm, travel and shopping addiction); and pain (including pain associated with migraine or headache or chronic pain).

As used herein, the term “treatment-resistant depression” or “TRD” means a depressive disorder which does not respond satisfactorily to adequate treatment. In embodiments, TRD includes a state of depression that persists after a course of antidepressant treatment including, for example, an inadequate response in a patient to at least one antidepressant course or trial of adequate dose(s) and duration, or as a failure of treatment to produce response or remission in a patient after two or more antidepressant treatment attempts of adequate dose and duration. TRD is a relatively common occurrence in clinical practice, with up to 50% to 60% of the patients not achieving adequate response following antidepressant treatment. TRD is a complex phenomenon influenced by variety in depressive subtypes, psychiatric comorbidity, and coexisting medical illnesses. Although TRD episodes are most commonly associated with major depressive disorder (MDD), it is also observed in other forms of depression as well as in the depressed phase of other disorders, such as bipolar disorder, that are associated with one or more depressive symptoms.

As used herein, the term “Tₘₐₓ” means the time to achieve maximum blood plasma concentration following administration. “Onset” as used herein, refers to the time period in which an active agent reaches a blood plasma concentration in an amount that elicits the first evidence of its effect, and may also be described as “onset of action”. A “rapid onset” in the context of the present disclosure means that the drug achieves Cₘₐₓ within a few minutes (e.g. about 2 minutes) of administration (e.g., by IV administration). A “delayed onset” in the context of the present disclosure means that the drug only achieves Cₘₐₓ after at a period of time that is longer than is typically required to observe onset of action for the particular active being administered via a typical route of administration. In some non-limiting embodiments a delayed onset can include a delay from several minutes to an hour or more (e.g., about 30, 40, 50, 60, 70, 80, 90, 100, 110, 120, 130, 140, 150, 160, 170, 180, 190, or 200 minutes or more) depending on dosage form and route of administration.

As used herein, the term “offset” means the time at which the drug plasma concentration reduces to a threshold level below which the drug no longer has any meaningful therapeutic effect. A “rapid offset” as used herein means the drug achieves this offset threshold in less than about 10 minutes following Tₘₐₓ. In some embodiments, a “delayed offset” as used herein means the delayed offset of the drug can include a delay from several minutes to an hour or more (e.g., about 30, 40, 50, 60, 70, 80, 90, 100, 110, 120, 130, 140, 150, 160, 170, 180, 190, or 200 minutes or more) depending on dosage form and route of administration.

### ASPECTS AND EMBODIMENTS OF THE DISCLOSURE

In one aspect, the present disclosure provides a pharmaceutical composition comprising Salvinorin A or derivative of Salvinorin A, wherein following administration (e.g. buccally or by subcutaneous injection) to a human subject, the Salvinorin A or Salvinorin A derivative human blood plasma Tₘₐₓ is between from about 10 minutes to about 240 minutes. In embodiments, Tₘₐₓ can comprise about 10, 20, 30, 40, 50, 60, 70, 80, 90, 100, 110, 120, 130, 140, 150, 160, 170, 180, 190, 200, 210, 220, 230, or 240 minutes. In some embodiments Tₘₐₓ can fall within a range comprising about 10 to about 30, 40, 50, 60, 70, 80, 90, 100, 110, 120, 130, 140, 150, 160, 170, 180, 190, 200, 210, 220, 230, or 240 minutes. In embodiments, Tₘₐₓ can comprise a range of from about 10 minutes to about 240 minutes, about 10 minutes to about 180 minutes, about 10 minutes to about 120 minutes, or about 10 minutes to about 60 minutes.

In one aspect, the disclosure provides a pharmaceutical composition comprising Salvinorin A or derivative of Salvinorin A, wherein following administration (e.g. buccally or by subcutaneous injection) to a human subject, the Salvinorin A or Salvinorin A derivative human blood plasma Tₘₐₓ is between from about 10 minutes to about 240 minutes, and after Tₘₐₓ is achieved, the concentration of Salvinorin A or derivative of Salvinorin A is maintained at about 50% or more of Cₘₐₓ for between about 10 minutes to about 90 minutes (e.g., about 10, about 15, about 20, about 25, about 30, about 35, about 40, about 45, about 50, about 55, about 60, about 65, about 70, about 75, about 80, about 85, or about 90 minutes).

In one aspect, the disclosure provides a pharmaceutical composition comprising Salvinorin A or derivative of Salvinorin A, wherein following administration (e.g. buccally or by subcutaneous injection) to a human subject, the Salvinorin A or Salvinorin A derivative human blood plasma Tₘₐₓ is between from about 10 minutes to about 240 minutes, and after Tₘₐₓ is achieved, the concentration of Salvinorin A or derivative of Salvinorin A is maintained at about 80% or more of Cₘₐₓ for between about 10 minutes to about 45 minutes (e.g. about 10, about 15, about 20, about 25, about 30, about 35, about 40 minutes, or about 45 minutes).

In one aspect, the present disclosure provides a pharmaceutical composition comprising Salvinorin A or derivative of Salvinorin A, wherein following administration (e.g. buccally or by subcutaneous injection) to a human subject, therapeutically effective blood levels of Salvinorin A or Salvinorin A derivative are maintained for at least about 20 minutes or longer (e.g., about 20, about 25, about 30, about 35, about 40, about 45, about 50, about 55, about 60, about 65, about 70, about 85, about 90, about 95, about 100, about 105, about 110, about 115, about 120, about 125, about 130, about 135, about 140, about 145, about 150, about 155, about 160, about 165, about 170, about 185, about 190, about 195, about 200, about 205, about 210, about 215, about 220, about 225, about 230, about 235, or about 240 minutes). In some embodiments of this aspect, therapeutically effective blood levels of Salvinorin A or Salvinorin A derivative are maintained for a time within a range of about 20 minutes to about 240 minutes, about 20 minutes to about 180 minutes, about 20 minutes to about 150 minutes, about 20 minutes to about 120 minutes, about 20 minutes to about 100 minutes, about 20 minutes to about 80 minutes, about 20 minutes to about 60 minutes, or about 20 minutes to about 40 minutes. In some further embodiments, therapeutically effective blood levels of Salvinorin A or Salvinorin A derivative are maintained for a time within a range of about 30 or 40 minutes to about 240 minutes, about 30 or 40 minutes to about 180 minutes, about 30 or 40 minutes to about 150 minutes, about 30 or 40 minutes to about 120 minutes, about 30 or 40 minutes to about 100 minutes, about 30 or 40 minutes to about 80 minutes, about 30 or 40 minutes to about 60 minutes, or about 30 minutes to about 40 minutes). In one embodiment of this aspect, blood levels of Salvinorin A or Salvinorin A derivative are maintained at a concentration that is below a concentration likely to cause undesirable side effects, such as memory loss.

In one aspect, the present disclosure provides a pharmaceutical composition comprising Salvinorin A or derivative of Salvinorin A, wherein following administration (e.g. buccally or by subcutaneous injection) to a human subject, at least 50% (e.g. about 50%, about 55%, about 60%, about 65%, about 70%, or about 75%) of the drug is released from the composition between about 10 minutes to about 60 minutes, about 10 minutes to about 50 minutes, about 10 minutes to about 40 minutes, or about 10 minutes to about 30 minutes.

In one embodiment of any of the previous aspects, the human blood plasma T½ is about 15 minutes to about 240 minutes, about 15 minutes to about 220 minutes, about 15 minutes to about 200 minutes, about 15 minutes to about 180 minutes, about 15 minutes to about 150 minutes, about 15 minutes to about 120 minutes, about 15 minutes to about 100 minutes, or about 15 minutes to about 90 minutes. In some further embodiments, T½ is about 20 minutes, about 30 minutes, about 40 minutes, about 50 minutes, about 60 minutes, about 70 minutes, about 80 minutes, or about 90 minutes..

In one embodiment of any of the previous aspects, the pharmaceutical composition comprises Salvinorin A.

In one embodiment of any of the previous aspects, the pharmaceutical composition is administered buccally. In a particular embodiment, the drug is absorbed across the buccal mucosa.

In one embodiment of any of the previous aspects, the pharmaceutical composition is administered by subcutaneous injection.

In embodiments of any of the previous aspects, Tₘₐₓ is 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 210, 220, 230, or 240 minutes.

In an aspect, the disclosure provides a buccal pharmaceutical composition comprising Salvinorin A or derivative of Salvinorin A, and a buccal film matrix comprising a solvent, a co-solvent, a surfactant, and a polymer in accordance with the embodiments described herein. In some embodiments the film matrix further comprises a permeability enhancer and/or an oil.

In some further embodiments, the polymer comprises copovidone, povidone, or hydroxypropyl cellulose or a combination thereof.

In some further embodiments, the solvent comprises N-methylpyrrolidone, methanol, ethanol, methyethylketone, or acetone or a combination thereof. In some further embodiments the solvent comprises N-methylpyrrolidone, polyethylene glycol or methanol or a combination thereof.

In some further embodiments, the surfactant comprises Lauroyl Polyoxyl-32 glycerides (e.g., Gelucire®), D-α-tocopheryl polyethylene glycol succinate or a combination thereof.

In some further embodiments, the permeability enhancer comprises L-menthol, chlorophyll, camphor, borneol, or a combination thereof.

In some further embodiments, the co-solvent comprises propylene glycol or benzyl alcohol or a combination thereof.

In some embodiments, the buccal pharmaceutical composition comprises Salvinorin A in an amount of about 0.01% to about 10% (%w/w) (e.g., about 0.01%, 0.1%, 1.0%, 2.0%, 3.0%, 4.0%, 5.0%, 6.0%, 7.0%, 8.0%, 9.0%, or about 10.0% w/w). In some embodiments, the buccal pharmaceutical composition comprises Salvinorin A in an amount (% w/w) of about 0.01%, 0.015%, 0.05%, 0.1%, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, or about 10.0% of the total weight of the dry buccal film.

In one aspect, the disclosure provides an injectable subcutaneous pharmaceutical composition comprising Salvinorin A or derivative of Salvinorin A, and a vehicle comprising a solvent, surfactant, and/or a co-solvent in accordance with the embodiments described herein.

In some embodiments, the solvent comprises a polar aprotic solvent or an oil or a combination thereof. In some further embodiments, the solvent comprises a pyrrolidone or an oil or a combination thereof. In some further embodiments, the pyrrolidone comprises N-methyl pyrrolidone (NMP).

In some embodiments, the co-solvent comprises a polyethylene glycol. In some further embodiments the polyethylene glycol comprises PEG300 or PEG400 or a combination thereof.

In some embodiments, the surfactant comprises a fatty acid ester. In some further embodiments the fatty acid ester comprises caprylocaproyl macrogolglycerides (e.g., Labrasol@).

In some embodiments, the vehicle further comprises a complexing agent comprising a cyclodextrin. In some further embodiments, the cyclodextrin comprises 2-hydroxypropyl-beta-cyclodextrin.

In some embodiments, the injectable subcutaneous pharmaceutical composition comprises Salvinorin A in an amount of about 0.01% to about 10% (%w/v) (e.g., about 0.01%, 0.1%, 1.0%, 2.0%, 3.0%, 4.0%, 5.0%, 6.0%, 7.0%, 8.0%, 9.0%, or about 10.0% w/v).

In some embodiments the vehicle further comprises a stabilizer, surfactant, precipitation inhibitor, preservative, and/or an antioxidant.

In some further embodiments of any of the above aspects and embodiments, the pharmaceutical compositions provide for improved stability of Salvinorin A or derivative of Salvinorin A.

In some further embodiments of any of the above aspects and embodiments, the pharmaceutical compositions provide for improved solubility of Salvinorin A or derivative of Salvinorin A.

In one aspect, the present disclosure provides a method of treating a neurological disease or condition, without inducing significant side effects, comprising administering buccally to a human subject an effective amount of Salvinorin A or a derivative of Salvinorin A. In one embodiment of this aspect, the drug is absorbed across the buccal mucosa.

In one aspect, the present disclosure provides a method of treating a neurological disease or condition, without inducing significant side effects, comprising administering by subcutaneous injection to a human subject an effective amount of Salvinorin A or a derivative of Salvinorin A.

### Pharmaceutical Compositions and Delivery

The formulation of Salvinorin compounds, such as Salvinorin A, is challenging due to the poor aqueous solubility of such compounds. However, various techniques can be utilized to formulate/solubilize the drug, including, but not limited to the preparation and use of microemulsions, nanoemulsions, polymeric nanoparticles, polymeric micelles, nano-structured lipid carriers, liposomes, and transfersomes. Solubility may also be enhanced by complexation, and by utilizing suitable surfactants and/or organic solvents alone or with water.

Solubility enhancements relevant to the preparation of formulations for buccal use (e.g., films) may include, for example, complexation with cyclodextrins, the use of organic solvents alone or in combination with water, and/or surfactants. The solubility studies of Salvinorin A described in the Examples have been carried out in various surfactants, polymers, plasticizers and oils, and have been followed up by compatability studies with Salvinorin A and various formulary components commonly present in films (see, e.g., Example 1).

### Compositions for Buccal Administration

Relative to an oral dosage form such as a tablet or capsule, buccal delivery can also provide for rapid absorption, faster onset of therapeutic action and avoidance of liver or gut wall first pass metabolism. For patients who have difficulty in swallowing tablets, capsules or other solids or those who have intestinal failure, the buccal delivery route is preferred.

Compositions for buccal administration include Salvinorin A or a derivative of Salvinorin A and at least one excipient to form a solid dosage form. The solid dosage form disintegrates in an oral cavity with minimal liquid exposure and at body temperature, and ideally adheres to the body tissue of the oral cavity via direct adhesion to tissue or buccal mucosa or entrapment of the dosage form in-between the gum and inner cheek. The solid dosage form disintegrates or melts in the oral cavity at body temperature with or without the aid of fluids, salivary fluids, mechanical erosion, or combinations thereof. Alternatively, the dosage form can be sprayed into the oral cavity in the form of a solution spray or a dry powder. Generally, the composition can be adhesive towards the body tissue lining the patient’s oral cavity.

The dosage form can be, but is not limited to, rapidly / fast dissolving and orodispersible tablets, bioadhesive patches or films, single layer film, bilayer film, multilayered film, sponges, lozenges, hard candies, wafers, disks, powders, ointments, pastes, emulsions, lollipops, mouthwashes, aerosols, sprays, gels, gummies, drops, microporous hollow fibers, tablets, bi-layer or multi-layer tablets, mucoadhesive tablets, gums, pills, pellets, spheres, or combinations thereof, and other forms known to those of skill in the art that are retained on the buccal mucosal surface.

Buccal films can be divided into three general categories based on time to release drug in the oral cavity. Quick release (QR) films completely dissolve in less than a minute, often within seconds, to rapidly release drug into the oral cavity. Moderate release films completely dissolve in a few minutes to up to about 20 minutes. Sustained release (SR) films dissolve more slowly than moderate release films, taking to up to about a few hours to completely dissolve. As moderate release and SR films offer longer contact times at the mucosal surface, the active ingredient is more likely to directly absorb through mucosa from a moderate release or SR film than from a QR film. Consequently, one particular embodiment of the present disclosure provides a moderate release buccal film comprising Salvinorin A or a derivative of Salvinorin A. Another particular embodiment of the present disclosure provides a SR buccal film comprising Salvinorin A or a derivative of Salvinorin A.

Pharmaceutical compositions of the present disclosure suitable for buccal administration may include one or more excipients, diluents, binders, lubricants, glidants, disintegrants, desensitizing agents, emulsifiers, mucosal adhesives, solubilizers, suspension agents, viscosity modifiers, ionic tonicity agents, buffers, carriers, surfactants, or mixtures thereof. Pharmaceutical compositions of the present disclosure suitable for buccal administration may also include components such as surfactant, co-surfactants, emulsifiers, oils, solvents, co-solvents, permeation enhancers, plasticizer, antioxidant, buffering agent, matrix polymers, mucoadhesive or bioadhesive polymers, and means for providing modified release, such as sustained or moderate release, of the active ingredient. The compositions can also include one or more pharmaceutically acceptable wetting agent, saliva stimulating agent, coloring agent, hydrophilic adjuvant / additive flavoring or other taste-masking agents.

Suitable mucoadhesive polymers include one or more polymers selected from cellulose derivatives, polyacrylic acids, polyacrylates, polyethylene oxides, polyvinyl pyrrolidones, povidones, copovidones, polyvinyl alcohols, tragacanth, alginates, gum (including karaya gum, guar gum, xanthan gum), soluble starch, gelatin, lectin, pectin, and chitosan. In some embodiments, the mucoadhesive polymer comprises one or more polymers selected from a hydrophilic polymer, a polysaccharide and its derivatives, and a hydrogel. In some embodiments, the mucoadhesive polymer comprises one or more polymers selected from polyacrylic acids, polyacrylates, celluloses, e.g., carboxycelluloses (e.g., sodium carboxymethyl cellulose), hydroxyalkyl cellulose (e.g, hydroxypropylcellulose, hydroxyethylcellulose and hydroxyethyl ethyl cellulose), polyvinylpyrrolidone, and polyvinyl alcohol. In some embodiments, the mucoadhesive polymer comprises one or more polymers selected from Carbopol (polyacrylic acid), carboxymethyl cellulose, carboxyethyl cellulose, hydroxypropyl cellulose, hydroxypropylmethyl cellulose, and gum. In some embodiments, the mucoadhesive polymer is water-swellable. Typically, the mucoadhesive polymer is present in an amount of about 15% to about 90% by weight of the film composition.

Suitable surfactant/co-surfactant and/or permeation enhancers may be selected from bile salts such as sodium deoxycholate (SDC), including sodium glycodeoxycholate (SGDC) and sodium taurodeoxycholate (STGC), synthetic surfactants such as cetylpyridinium chloride (CPC), sodium lauryl sulfate (SLS) or a polyethoxylated sorbitan (e.g., Tween 80), L-menthol, dimethyl sulfoxide (DMSO), oleic alcohol, oleic acid, oleyl oleate, levulinic acid, propylene glycol, dipropyleneglycol, ethanol, and other surfactants. Other permeation enhancers may include 23-lauryl ether, aprontinin, azone, benzalkonium chloride, cetylpyridinium chloride, cetyltrimethylammonium bromide, cyclodextrin, dextran sulfate, lauric acid, lysophosphatidylcholine, sodium methoxysalicylate, methyl oleate, phosphatidylcholine, polyoxyethylene, polysorbate, sodium ethylenediaminetetraacetic acid, sodium glycocholate, sodium glycodeoxyocholate, sodium salicylate, sodium taurocholate, sodium taurodeoxycholate, short and medium chain mono-, di- and triglycerides and other polyol esters, and various alkyl glycosides, linoleoyl polyoxyl-6 glycerides, lauroyl polyoxyl-32 glycerides, D-α-tocopheryl polyethylene glycol succinate (Vitamin E TPGS / TPGS), chlorophyll, camphor, Borneol, and the like. In some embodiments, the surfactant/co-surfactant and/or permeation enhancers is present in an amount of about 0.5% to about 40% by weight of the film composition.

In some embodiments, the film composition comprises a solvent/solubilizer including, for example, non-limiting embodiments of benzyl alcohol, benzyl benzoate, N-mehtylpyrrilidone, polyethyleneglycols, glycerols, propylene glycol, and the like, or mixtures thereof. In some embodiments, a solvent/solubilizer is present in an amount of about 0.5% to about 10% by weight of the film composition.

In some embodiments, the film composition comprises an oil or fatty acid derivative including, for example, non-limiting embodiments of castor oil, peppermint oil sesame oil, medium chain tryglyceride, TPGS (soluble vitamin E), sodium deoxycholate, sodium glycodeoxycholate, sodium taurodeoxycholate hydrate, polyoxyethylene (20) oleyl ether (Oleth-20, Brij 020), Plasacryl (glycerol monostearate + triethyl citrate + polysorbate 80), PEG 35 castor oil, SPAN 60, SPAN 40, SPAN 40 Stearate, L-menthone, S-limonene, eucaliptol, soybean oil, oleyl alcohol, and the like, or mixtures thereof. In some embodiments, an oil or fatty acid derivative is present in an amount of about 0.5% to about 10% by weight of the film composition.

In some embodiments, the film composition comprises an antioxidant, e.g., comprising one or more antioxidants such as tocopherol acetate, L-glutahione, L-cysteine, butylated hydroxytoluene (BHT), butylated hydroxyanisole (BHA), tocobiol and ethylenediaminetetraacetic acid (EDTA).

In some embodiments, the SR film compositions can be combined, e.g., laminated, with a QR film to form a bilayer or multilayer film composition. Typically, such a bilayer or multilayer film can provide a bi-phasic release profile, which can be advantageous in certain situations. In some embodiments, the quick-release film layer comprises a water-soluble polymer. In some embodiments, the water-soluble polymer in the quick-release film layer comprises one or more polymers selected from hydroxyl propyl methyl cellulose (HPMC), hydroxylpropyl cellulose (HPC), Povidone, polyvinyl alcohols (PVA), low molecular weight polyethylene oxide, and starch-based polymers. In some embodiments, the QR film layer can also optionally include a permeation enhancer, e.g., one or more permeation enhancers selected from dimethyl sulfoxide (DMSO), oleic alcohol, oleic acid, oleyl oleate, levulinic acid, propylene glycol, dipropylene glycol, ethanol, and surfactants. In some embodiments, the QR film layer can also optionally include an antioxidant, such as tocopherol acetate.

There are numerous compositions and delivery vehicles suitable for buccal delivery of the active ingredients. In addition to Salvinorin A or a derivative of Salvinorin A, other components of dosage forms include, but are not limited to, starch, mannitol, kaolin, calcium sulfate, inorganic salts, such as sodium chloride, powdered cellulose derivatives, dibasic and tribasic calcium phosphate, calcium sulfate, magnesium carbonate, magnesium oxide, poloxamers such as polyethylene oxide, hydroxypropyl methylcellulose, anionic excipients, cationic excipients, zwitterionic excipients, polymeric hydrogel, powder microsphere mucoadhesive compositions, thiolated polymeric excipients, polycationic material, chitosan, cross-linked starches, fats, carbohydrates, polyols, buffers, phosphate buffers, acetate buffers, methocel, sodium chloride, water, lactic acid, benzalkonium chloride, demineralized water, cellulose, microcrystalline cellulose, hydroxypropyl cellulose, hydrogenated vegetable oil, flavoring agents, phospholipids, xylitol, cacao, combinations thereof, and other similar excipients known to those of skill in the art.

The film compositions can further include plasticizers. A plasticizer improves the flexibility of the film and reduces the brittleness of the film by reducing the film’s glass transition temperature. Examples of suitable plasticizers for use herein include one or more plasticizers selected from polyethylene glycols (PEGs) such as PEG 300 and PEG 400, propylene glycol, glycerol, triacetin and castor oil. In some embodiments, the plasticizer is present in an amount of about 0.1% to about 20% by weight of the film composition.

The film compositions can further include a sweetening agent, a saliva stimulating agent and/or a flavoring agent. An increase in saliva production can aid faster solubilization of the film resulting in faster drug absorption. A natural or artificial sweetener may be used to improve the palatability of the film. For example, in some embodiments, the film composition comprises a sweetener selected from, but not limited to, sucrose, dextrose, fructose, glucose, liquid glucose, maltose, saccharin, sucralose, neotame, cyclamate, aspartame, and acesulfame-K, and the like. In some embodiments, the film composition can further include a salivary stimulating agent such as, for example, the non-limiting embodiments of citric acid, malic acid, lactic acid, ascorbic acid or tartaric acid. Saliva stimulating agents can be used alone or in combination, and be present in amounts, for example, between about 0.1 to about 8% w/w of weight of the dry film composition. In some embodiments, the film composition can further include one or more flavoring agent such as, for example, the non-limiting embodiments of peppermint oil, cinnamon oil, vanilla extract, menthol, L-menthol, or combinations thereof.

A second layer may also be applied to the buccal film as a placebo layer (not containing drug), generally referred to as a backing layer, to limit the swallowing of drug by solubilizing and releasing the drug into the oral cavity. The backing layer also maximizes drug permeation through the buccal mucosa, and helps to maintain the desired microenvironmental pH intended for drug solubilization. The backing layer may conveniently comprise the same or similar excipients to the film layer containing drug.

Buccal films herein may be prepared, for example, by a standard solvent casting method or by a hot melt extrusion technique well known in the art. The solvent casting method is generally preferred, utilizing various solvents (one or more) to solubilize the drug and other excipients to form a homogenous mixture which is then cast into the film followed by a drying process. In the hot melt extrusion technique, the drug is co-melted with suitable polymers and excipients to form solid solution and extruded as a film. The films are then cut in to specified dimensions to produce individual doses.

The amount of active agent, e.g., Salvinorin A or a derivative thereof, to be incorporated into the buccal film depends on the desired dosage to be administered. In some embodiments, for example, Salvinorin A or a derivative thereof can be present in about 0.01% to about 10% by weight of film.

Buccal films were prepared with a thickness range from about 0.01 mm to about 1.5 mm, and more specifically from about 0.05 to about 0.4 mm. Further, the thickness of the film could be varied from 10% to 90% to these ranges based on the drug-polymeric mixture.

Buccal films were prepared with a loss on drying (LOD) range from about 2 to about 15% by film weight, and more specifically from about 5 to about 10% by film weight.

### Compositions for Administration Via Subcutaneous Injection

In some aspects and embodiments, an injectable pharmaceutical composition in accordance with the disclosure may include one or more excipients, diluents/vehicles, solvents, co-solvents, desensitizing agents, emulsifiers, solubilizers, suspension agents, viscosity modifiers, ionic tonicity agents, buffers, carriers, surfactants, cryoprotectants, lyoprotectants, antioxidant, chelating agent, inert gases, complexing agents, preservatives or mixtures thereof.

Suitable injectable formulations comprising Salvinorin A or a derivative of Salvinorin A for subcutaneous administration are predicated on solvent systems capable of solubilizing the drug. Development of SC formulations of Salvinorin A is challenging due to its poor solubility in different vehicles. Various techniques can be utilized to achieve complete solubilization of Salvinorin A at the site of absorption, including but not limited to microemulsion, nanoemulsions, polymeric nanoparticles, polymeric micelles, nanostructured lipid carriers, liposomes, transformers and the like, including those illustrated in Examples described hereinbelow.

Vehicles suitable for the preparation of stable Salvinorin A formulations for subcutaneous administration may include one or more of: ethanol, PEG 300 or 400, medium chain triglycerides, pegylated derivatives of medium chain fatty acid triglyceride of capric and caprylic acid (e.g. Labrasol), N-methylpyrrolidone, dimethyl sulfoxide, propylene glycol, benzyl benzoate, benzyl alcohol, castor oil, sesame oil, 5% lecithin in sesame oil, 30% sulfobutylether-(β-cyclodextrin (SBECD) in water, 30% 2-hydroxypropyl-beta-cyclodextrin (HPβCD) in water, 10% Tween 20 in water, 10% Tween 80 in water, 50% Tween 80 in water, 10% poloxamer 188 in water, 10% sodium deoxycholate in water, 10% cremaphor EL in water, 50% cremaphor EL in water, 2% surfactant (as described hereinabove) / 30% PEG 300 / water, 2% surfactant (as described hereinabove) / 30% PEG 300 / 30% cyclodextrin / water, 2% surfactant (as described hereinabove) / 30% PEG 300 / 30% cyclodextrin / water, 10% NMP / 90% PEG 300, 10% NMP / 45% PEG 300 / 45% cremaphor EL, 10% NMP / 30% PEG 300 / 30% cremaphor EL / 30% water, 30% NMP / 70% PEG300, 30% NMP / 70% PEG 400, 20% NMP / 80% PEG300, 20% NMP / 80% PEG 400, 10% NMP / 90% PEG300, 10% NMP / 90% PEG 400. In one embodiment, the vehicle comprises N-methylpyrrolidone and Labrasol. In one embodiment, the vehicle consists of N-methylpyrrolidone and Labrasol. In one embodiment, the vehicle consists of N-methylpyrrolidone, Labrasol and PEG 400. In one embodiment, the ratio of N-methylpyrrolidone to Labrasol is 1:9 v/v. In one embodiment, the ratio of N-methylpyrrolidone to Labrasol + PEG 400 is 1:9 v/v. In one embodiment, the ratio of N-methylpyrrolidone to Labrasol to PEG 400 is 1:4.5:4.5 v/v. In some embodiments, the vehicle comprises one or more of a solvent, co-solvent and surfactant, and is present in an amount of about 5% to up to about 99.99% by weight of the formulation composition.

The formulations herein for subcutaneous administration may also be pH adjusted, e.g. within the range of about pH 4 to about pH 9. Furthermore, the osmolarity will be set at not more than 600.

In some embodiments, pH modifiers and/or buffering agents for use in injectable formulations (e.g., subcutaneous administration) described herein include non-limiting examples of sodium hydroxide and hydrochloric acid, citric acid, sodium hydrogen phosphate, O-phosphoric acid, sodium phosphate - dibasic dihydrate disodium phosphatemonobasic, sodium phosphate -dibasic dodecahydrate, sodium dihydrogen phosphate monohydrate, disodium hydrogen phosphate dihydrate, sodium chloride, meglumine, and dibasic sodium phosphate anhydrous, or combinations thereof.

In some embodiments, preservatives for use in injectable formulations (e.g., subcutaneous administration) described herein include non-limiting examples of benzyl alcohol, chlorobutanol, benzalkonium chloride, chlorobutanol, phenoxyethanol, m-cresol, methyl paraben, propyl paraben, phenol, phenoxyethanol, thiomersal, sodium benzoate, benzoic acid, ethanol, or combinations thereof.

In some embodiments tonicity adjusting agents for use in injectable formulation (e.g., subcutaneous administration) herein include non-limiting examples of salts, such as halide salts of alkali and alkaline metals (e.g., sodium chloride, potassium chloride etc.), mannitol, glycerin, glucose, dextrose, or combinations thereof.

In one aspect, the injection formulations herein comprise one or more viscosity modifiers. Examples include sodium carboxymethylcellulose, hydroxypropyl methylcellulose, hydroxypropyl cellulose, hydroxyethyl cellulose, and polyvinyl pyrrolidone.

The finished formulation may be packaged as a solution in a single or multi-use clear or amber vial using a suitable closure, or as a prefilled syringe, or unit dose clear or amber ampuoles. Alternatively, the formulation may be freeze-dried and the lyophilized product reconstituted just prior to use.

The amount of active e.g., Salvinorin A or its suitable form, to be incorporated into the injectable formulation (e.g., subcutaneous injection formulation) at the desired dosage to be administered. For example, Salvinorin A or a derivative thereof can be present in about 0.01% to about 10%.

In some embodiments, the formulation can be formulated with vehicle that can increase or optimize solubilization of the active agent (i.e., Salvinorin A). In some embodiments the vehicle comprises 30% NMP / 70% PEG300; 30% NMP / 70% PEG 400; 20% NMP / 80% PEG300; 20% NMP / 80% PEG 400; 10% NMP / 90% PEG300; 10% NMP / 90% PEG 400, and can be present in an amount of about 5% to up to about 99.99% by weight of the formulation composition.

In some embodiments, the formulation can be prepared and stored under an inert atmosphere (e.g., nitrogen) which may help prolong storage stability of the formulation. As detailed below, stability studies under various conditions have been evaluated (e.g., at 2-8° C., 25° C. / 60% RH (upright orientation) and at 60° C. (upright and inverted orientations)). The sample stability can be analyzed by appearance, assays for impurity and/or degradation product detection level (i.e., HPLC methods). In some embodiments, storage-stable formulations demonstrate acceptable stability under various conditions for up to 4 or more weeks.

### DOSING

Salvinorin A may conveniently be administered in purified form, which can be obtained from commercial sources. Thus, for example, the purity may conveniently exceed 95%, such as 98% purity or greater. The dose of Salvinorin A or a derivative of Salvinorin A, when administered buccally or subcutaneously in a composition of the present disclosure, may conveniently be in a range of from about 100 µg to about 10,000 µg, e.g. from about 1,000 µg to about 5,000 µg, including 1,000 µg, 1,500 µg, 2,000 µg, 2,500 µg, 3,000 µg, 3,500 µg, 4,000 µg, 4,500 µg, 5,000 µg, 5,500 µg, 6,000 µg, 6,500 µg, 7,000 µg, 7,500 µg, and 8,000 µg. In an embodiment, a composition of the disclosure comprising Salvinorin A or a derivative of Salvinorin A may be administered buccally or subcutaneously at a dose of about 1.5 µg/kg to about 150 µg/kg, including about 1.5 µg/kg to about 5.0 µg/kg, about 1.5 µg/kg to about 10.0 µg/kg, about 1.5 ug/kg to about 15.0 ug/kg, about 1.5 µg/kg to about 20.0 µg/kg, about 1.5 µg/kg to about 25.0 µg/kg, about 1.5 µg/kg to about 30.0 µg/kg, 1.5 µg/kg to about 35.0 µg/kg, about 1.5 µg/kg to about 40.0 µg/kg, about 1.5 µg/kg to about 45.0 µg/kg, about 1.5 µg/kg to about 50.0 µg/kg, 1.5 µg/kg to about 55.0 µg/kg, about 1.5 µg/kg to about 60.0 µg/kg, about 1.5 µg/kg to about 65.0 µg/kg, about 1.5 µg/kg to about 70.0 µg/kg, 1.5 µg/kg to about 75.0 µg/kg, about 1.5 µg/kg to about 80.0 µg/kg, about 1.5 µg/kg to about 85.0 µg/kg, about 1.5 µg/kg to about 90.0 µg/kg, 1.5 µg/kg to about 95.0 µg/kg, about 1.5 µg/kg to about 100.0 µg/kg, about 1.5 µg/kg to about 105.0 µg/kg, about 1.5 µg/kg to about 110.0 µg/kg, 1.5 µg/kg to about 115.0 µg/kg, about 1.5 µg/kg to about 120.0 µg/kg, about 1.5 µg/kg to about 125.0 µg/kg, about 1.5 µg/kg to about 130.0 µg/kg, 1.5 µg/kg to about 135.0 µg/kg, 1.5 µg/kg to about 140.0 µg/kg, 1.5 µg/kg to about 145.0 µg/kg, or about 1.5 µg/kg to about 150.0 µg/kg. In a further embodiment, a composition of the present disclosure comprising Salvinorin A or a derivative of Salvinorin A may be administered buccally or subcutaneously at a dose of about 1.5 µg/kg to about 16.5 µg/kg, including about 1.5 µg/kg to about 4.5 µg/kg, about 1.5 µg/kg to about 6.0 µg/kg, about 1.5 ug/kg to about 7.5 ug/kg, about 1.5 µg/kg to about 9.0 µg/kg, about 1.5 µg/kg to about 10.5 µg/kg, about 1.5 µg/kg to about 12.0 µg/kg, 1.5 µg/kg to about 13.5 µg/kg or about 1.5 µg/kg to about 15.0 µg/kg.

Administration may be once a day (q.d.), twice a day (b.i.d.), three times a day (t.i.d.), four times a day (q.i.d.) or at more or less frequent intervals such as once every other day (q.a.d.), once every third day, twice a week (bis in 7 d.), once a week (QWK), once every other week, etc. Alternatively, administration may be as needed (p.r.n.).

### USES

In one aspect, the present disclosure provides a pharmaceutical composition, as further described herein, for use in treating a neurological disease or condition.

In one aspect, the present disclosure provides a method of treating a neurological disease or condition, comprising administering buccally or by subcutaneous injection to a human subject an effective amount of Salvinorin A or derivative of Salvinorin A.

In one aspect, the present disclosure provides a method of treating a neuropsychiatric disorder, comprising administering buccally or by subcutaneous injection to a human subject an effective amount of Salvinorin A or derivative of Salvinorin A.

Examples of neuropsychiatric disorders which may be treated with Salvinorin A or a derivative of Salvinorin A include depression, treatment-resistant depression (TRD), anxiety, bipolar disorder, post-traumatic stress disorder, abnormalities of mood or emotion, including the above conditions, dysthymia, schizoaffective disorder, schizophrenia and other psychotic disorders, panic disorder, traumatic stress disorders, phobic disorders, and personality disorders with abnormal mood, such as borderline personality disorder, schizoid and schizotypal disorders and suicide ideation, or rumination/unproductive repetitive thoughts negatively impacting one’s behavior/mood/ability to focus.

In one aspect, the present disclosure provides a method of treating addiction, comprising administering buccally or by subcutaneous injection to a human subject an effective amount of Salvinorin A or derivative of Salvinorin A.

Examples of addiction which may be treated with Salvinorin A or a derivative of Salvinorin A include substance use disorder such as addiction to nicotine, alcohol, cocaine, opioids, amphetamine, methamphetamine, heroin, morphine, phencyclidine, 3,4-methylenedioxymethamphetamine, as well as other addictive substances.

In one aspect, the present disclosure provides a method of treating addictive behavior, comprising administering buccally or by subcutaneous injection to a human subject an effective amount of Salvinorin A or derivative of Salvinorin A.

Examples of addictive behavior which may be treated with Salvinorin A or a derivative of Salvinorin A include addiction to eating, gambling, sex, pornography, videogames, work, exercise, spiritual obsession, self-harm, travel and shopping.

In one aspect, the present disclosure provides a method of treating pain, comprising administering buccally or by subcutaneous injection to a human subject an effective amount of Salvinorin A or derivative of Salvinorin A.

Examples of pain which may be treated with Salvinorin A or a derivative of Salvinorin A include pain associated with migraine or headache or chronic pain.

Compositions of the present disclosure may be administered to effectively treat the disorders and conditions heretofore described, without also inducing the undesirable side effects which Salvinorin A is known to produce when given intravenously and by inhalation.

Thus, in one embodiment, the present disclosure provides a method of treating a neurological disease or condition, without also inducing significant side effects, comprising administering buccally or by subcutaneous injection to a human subject an effective amount of Salvinorin A or derivative of Salvinorin A.

In a further embodiment, the present disclosure provides a method of treating a neurological disease or condition, without also inducing significant side effects, such as memory loss or impairment of motor skills, comprising administering buccally or by subcutaneous injection to a human subject an effective amount of Salvinorin A or derivative of Salvinorin A.

Compositions of the present disclosure may be especially suitable to treat human subjects prone to neuropsychiatric disorders due to their genetic make-up. Thus, for example, humans highly susceptible to depressive episodes due to their genetic make-up, e.g. subjects having a rs1051660 or RS16918875 SNP mutation in an opioid receptor kappa 1 (OPRK1) gene, may represent particular candidates for treatment with a composition of the present disclosure.

### COMBINATION THERAPY

The methods described herein include administering Salvinorin A or a derivative of Salvinorin A as the sole active ingredient. However, also encompassed within the scope of the present disclosure are methods for treating a neurological disease or condition that comprise administering Salvinorin A or a derivative of Salvinorin A in combination with one or more additional agents. In one aspect, these additional agents are therapeutic agents appropriate for the disease or disorder that is being treated, as is known in the art. Examples include agents that increase neuroplasticity, such as valproic acid or beta-hydroxybutyrate. In another aspect, these additional agents may be inhibitors of enzymes that can metabolize Salvinorin A or a derivative of Salvinorin A following administration, such as CYP enzymes. Examples of suitable CYP inhibitors that can be used to extend the time Salvinorin A or a derivative of Salvinorin A is present at therapeutic levels in the human body include inhibitors of UGT2B7 (e.g., flunitrazepam, indomethacin, chenodeoxycholic acid, ketoconazole, dovitnib, eltrombopag, umifenovir), CYP2D6 ( e.g., bupropion, fluoxetine, paroxetine, quinidine, terbinafine, amiodarone, celecoxib, fluvoxamine, labetalol, ritonavir, sertraline, vemurafenib), CYP1A1 (e.g., hesperetin, rhapontigenin, α-Naphthoflavone, rutaecarpine, pterostilbene), CYP2C18 (sulfaphenazole), CYP2E1 (e.g., rufinamide, nifedipine, ticlopidine, nabilone, zucapsaicin, rhein, clotrimazole, isoniazid, disulfiram, midostaurin), or p-glycoprotein (e.g., amiodarone, clarithromycin, erythromycin, propafenone, quinidine, azithromycin, ketoconazole, cyclosporine, or combinations thereof. In another aspect, these additional agents may be esterase inhibitors (e.g. inhibitors of serine esterases and carboxyl esterases).

The further agent(s) may be incorporated into the same composition as Salvinorin A or a derivative of Salvinorin A, or may be administered as a separate composition. When administered as a separate composition, the further agent(s) may be given by the same route as Salvinorin A or a derivative of Salvinorin A or by a different route. The further agent(s) may also be administered prior to, during and/or after the administration of Salvinorin A or a derivative of Salvinorin A. Dosage regimens may be adjusted to provide the optimum desired response. Treatment dosages may be titrated using routine methods known to those of skill in the art to optimize safety and efficacy.

In a further aspect of the present disclosure, when treating a neuropsychiatric disease or disorder, such as depression, treatment-resistant depressison (TRD), anxiety or an addiction, compositions of the present disclosure may be administered in conjunction with psychotherapy, talk therapy, cognitive behavioral therapy, exposure therapy, biofeedback therapy (e.g. EEG-assisted therapy and virtual reality assisted therapy), systematic desensitization, mindfulness, dialectical behavior therapy, interpersonal therapy, eye movement desensitization and reprocessing, social rhythm therapy, acceptance and commitment therapy, family-focused therapy, psychodynamic therapy, light therapy, computer therapy (including digital cognitive behavioral therapy), cognitive remediation, exercise, or other types of therapy such as transcranial magnetic stimulation (TMS). In one embodiment, compositions of the present disclosure may be administered to treat depression in conjunction with digital cognitive behavioral therapy, for example, using the digital program DEPREXIS®. In one embodiment, compositions of the present disclosure may be administered (for example, to treat depression or anxiety) in conjunction with therapy using a transdiagnostic approach (cf. J Consult Clin Psychol. 2020 Mar;88(3): 179-195).

All references, articles, publications, patents, patent publications, and patent applications cited herein are incorporated by reference in their entireties for all purposes. However, mention of any reference, article, publication, patent, patent publication, and patent application herein is not, and should not be, taken as acknowledgment or any form of suggestion that they constitute valid prior art or form part of the common general knowledge in any country in the world.

### EXAMPLES

The following examples are presented in order to more fully illustrate certain aspects and embodiments of the disclosure. They should in no way be construed, however, as limiting to the scope of the appended claims.

### Example 1 Salvinorin A Solubility and Film Formulations

### Example 1.1. Initial Salvinorin A Solubility and Compatability Assessment

The various solutions are prepared from supersaturated mixtures and analyzed using HPLC. The results are shown in Tables 1 and 2 below. Alternative or further surfactants which may be added to improve solubility include Kollidon 12, Kollidon 17, Kolliphor, Polyoxyl 35 castor oil, Poloxomer 188 and Poloxamer 407. Depending upon the desired release profile, Salvinorin A can also be added following milling or micronization, or in the form of nano-crystals or nanoparticles. Compatibility studies with Salvinorin A and common buccal film formulary components are shown in Table 3.

TABLE 1

Solubilization of Salvinorin A mixed with solubilizers in water and oil

| Solubilizer | solubilizer (mg) | Salvinorin A (mg) | water (ml) | Solubility (mg/ml) HPLC |
|---|---|---|---|---|
| Deionised water | 0 | 21 | 10 | 0.000 |
| polysorbate 80 (Tween 80) | 50 | 21 | 10 | 0.000 |
| Poloxamer 407 (Lutrol micro 127 MP) | 150 | 21 | 10 | 0.008 |
| Povidone (PVP K-90) | 200 | 23 | 10 | 0.000 |
| Copovidone (Plasdone S-630) | 200 | 22 | 10 | 0.000 |
| polysorbate 20 (Tween 20) | 50 | 22 | 10 | 0.000 |
| Triacetin | 200 | 21 | 10 | 0.000 |
| PEG 300 | 150 | 21 | 10 | 0.000 |
| PEG 400 | 150 | 21 | 10 | 0.000 |
| TPGS (soluble vitamin E) | 100 | 23 | 10 | 0.011 |
| Propylene glycol | 150 | 22 | 10 | 0.000 |
| Sodium dodecyl sulfate (SDS; SLS) | 50 | 21 | 10 | 0.013 |
| Benzalkonium chloride (BKC) | 50 | 21 | 10 | 0.008 |
| Cetylpyridinium chloride (CPC) | 50 | 23 | 10 | 0.000 |
| Sodium Deoxycholate | 100 | 22 | 10 | 0.078 |
| Sodium Glycodeoxycholate | 100 | 1 | 10 | 0.000 |
| Sodium Taurocholate hydrate | 100 | 21 | 10 | 0.000 |
| Sodium Taurodeoxycholate hydrate | 100 | 21 | 10 | 0.000 |
| alpha-cyclodextrin (cavamax W6) | 200 | 23 | 10 | 0.000 |
| beta-cyclodextrin (cavamax W7) | 180 | 22 | 10 | 0.000 |
| gamma-cyclodextrin (cavamax W8) | 200 | 21 | 10 | 0.000 |
| Hydroxypropyl-gamma-cyclodextrin(cavasol W8 HP) | 200 | 22 | 10 | 0.000 |
| Polyoxyethylene (20) oleyl ether (Oleth- 20, Brij 020) | 200 | 21 | 10 | 0.000 |
| Plasacryl (Glycerol monostearate + Triethyl citrate+polysorbate 80) | 192 | 21 | 10 | 0.000 |
| Soy lecithin | 103 | 10 | 5 | 0.000 |
| PEG 35 castor oil | 110 | 11 | 5 | 0.000 |
| SPAN 60 | 103.00 | 12.00 | 5 | 0.013 |
| SPAN 40 | 104.00 | 12.00 | 5 | 0.000 |
| SPAN 40 Stearate | 110.00 | 11.00 | 5 | 0.000 |

TABLE 2

Salvinorin A in solubilizer oils

| Solubilizer | solubilizer (g) | Salvinorin A (mg) | Solubility (mg/ml) HPLC |
|---|---|---|---|
| L Menthone | 5 | 10.00 | 0.101 |
| S Limonene | 5 | 10.00 | 0.052 |
| Peppermint oil | 5 | 9.90 | 0.384 |
| Eucaliptol | 5 | 12.00 | 0.108 |
| Soybean oil | 5 | 11.00 | 0.032 |
| Oleyl Alcohol | 5 | 11.00 | 0.065 |

TABLE 3

Compatibility of Salvinorin A and Formularies (50° C. / 75% RH; HPLC detection)

| Binary mixture | # of Impurity | Impurity % |
|---|---|---|
| Salvinorin A + Control | 0 | 0 |
| Salvinorin A + Methyl ethyl ketone (MEK) | 1 | 0.85 |
| Salvinorin A + Butylated hydroxytoluene (BHT) | 1 | 0.83 |
| Salvinorin A + Methanol | 1 | 0 |
| Salvinorin A + Ammoniun glycyhrrhizate | 2 | 30.12/4.28 |
| Salvinorin A + Sucralose | 0 | 0.00 |
| Salvinorin A + Titanium dioxide | 1 | 25.57 |
| Salvinorin A + Plasdone S630 | 3 | 1.00/0.79/2.70 |
| Salvinorin A + low substituted hydroxypropyl cellulose (L-HPC) | 1 | 0.73 |
| Salvinorin A + Triacetin | 4 | 2.56/5.07/1.17/2.16 |
| Salvinorin A + PEG300 | 0 | 0 |
| Salvinorin A + Maltitol | 1 | 0.79 |
| Salvinorin A + PVP k90 | 3 | 0.76/1.14/0.98 |
| Salvinorin A + HMPC E5 | 0 | 0 |
| Salvinorin A + Polyethylene Oxide | 0 | 0 |

The solubility and compatability studies are used as a basis for the design of the film and injectable formulations, considering the overall solubility of Salvinorin A and stability of the components in the formulations.

### Example 1.2. Initial Film Formulations

A series of film formulations for oral, buccal, or sublingual administration are prepared and evaluated for various parameters following administration. Composition of film-based formulations can include the components and ranges in Table 4.

TABLE 4

Initial Buccal Film Formulations

| Ingredient | Formulation 1 | Formulation 2 | Formulation 3 | Formulation 4 | Formulation 5 |
|---|---|---|---|---|---|
| Salvinorin A | 0.5-3 | 0.5 | 1 | 1.5 | 2 |
| Hydroxypropyl cellulose | 25-70 | 53 | - | - | 22 |
| Polyethylene oxide | 20-70 | | 54 | - | 22 |
| Hypromellose | 2-20 | 15 | 8 | 15 | 11 |
| Hydrophilic polymer | 10-70 | 14 | 20 | 61.5 | 20 |
| Surfactant/solubilizer | 1-10 | 2 | 1 | 2 | 5 |
| Polyethylene glycol PEG300/400 | 1-6 | 4 | 3 | - | 2.5 |
| Propylene glycol | 1-6 | - | - | 3 | - |
| Oil (e.g. castor oil) | 0-8 | - | - | 3 | 2 |
| Sucralose | 0.5-5 | 2 | 2 | 2 | 2 |
| Antioxidant | 0.1-5 | 1 | 2 | 1 | 1.5 |
| Peppermint oil | 0.1-2 | 1 | 1 | 1 | 1 |
| Citric acid | 0.2-15 | 7.5 | 8 | 10 | 5 |
| Acetone (% of wet film) | 10-80 | 50 | - | 60 | 30 |
| Water (% of wet film) | 2-40 | 25 | 15 | 10 | 10 |
| Isopropyl alcohol (% of wet film) | 10-80 | - | 60 | - | 30 |

Note: all ingredient amount is in % w/w to dry film except solvents

Additional embodiments of film formulations can be prepared in accordance with the embodiments of formulation 6, detailed in Table 5, below.

TABLE 5

Buccal Formulation

| Ingredients (Formulation 6) | % (w/w) Dry film Composition |
|---|---|
| Salvinorin A | 0.01-10 |
| Polymer/s | 15-90 |
| Surfactant/solubilizer | 0.5-15 |
| Co-surfactant | 0.5-10 |
| Plasticizer | 0.1-20 |
| Oil | 0.5-10 |
| Solvent / co-solvent | 0.5-10 |
| Sweetener | 0-5 |
| Flavoring Agent | 0-3 |
| Permeability Enhancer | 0.5-10 |
| Organic Solvent/s (e.g., Methanol, Methyl ethyl Ketone, Acetone, isopropyl alcohol etc. or its mixture - % of solvent composition) | 50-100 |
| Water (% of solvent composition) | 0-50 |

Films are prepared as formulations 1-6 and evaluated for film apperance, film solubility and dissolution characteristics. Characterization parameters of the films can include folding endurance, tensile strength, % elongation, content uniformity, disintegration time, potency, and dissolution. Ex-vivo permeation studies are also performed on the films and modelled to film performance in animals and humans. Ex-vivo permeation studies can be conducted using Franz diffusion cells or Ussing chambers utilizing animal mucosa (e.g. pig or sheep buccal mucosa) or using commercially available synthetic membranes (e.g. Permeapad®).

The Franz diffusion cell consists of two compartments: one is a donor compartment and the other is a receptor compartment of 18 mL capacity and having 0.785 cm2 effective diffusion area. The temperature is maintained at 37° C. by a water jacket. This technique is used to establish the optimal level of permeation enhancer in the formulation and determine the time required for the film to be retained at the buccal surface to provide the desired rate and extent of drug absorption.

Permeapad® (Certificate No. 014557268) is a barrier consisting of a support layer and lipid layer. The barrier lipid layer comprises soy phosphatidylcholine S-100. A thin layer of lipid is applied to a hydrophilic support sheet (Pütz GmbH, Taunusstein, Germany) in organic solution. The solvent is allowed to evaporate to form the barrier. The permeability of the test formulation is determined across the Permeapad®. In other studies, Permeapad® has been shown to be a predictive assay for pH dependent permeability and useful as a preliminary permeability tool for buccal absorption. (See, Hanady Ajine Bibi et al., European Journal of Pharmaceutical Sciences 93 (2016) 399-404).

### Example 1.3 Additional Salvinorin A Films

A further series of Salvinorin A loaded buccal films are prepared as described in Table 6 and Table 7.

TABLE 6

Salvinorin A Film Formulations

| Ingredient | Formulation 7 | Formulation 8 |
|---|---|---|
| | Composition (% w/w) | |
| Salvinorin A | 1.37 | 2.42 |
| Copovidone | 24.27 | 23.54 |
| Hydroxypropyl Cellulose | 48.49 | 47.06 |
| Gelucire 44/14 | 8.72 | 8.10 |
| Linoleoyl Polyoxyl-6 glycerides | 3.59 | 3.14 |
| Polyethylene glycol,300 | 4.33 | 4.11 |
| Propylene glycol | 2.49 | 5.08 |
| N-methylpyrrolidone | 4.23 | 4.11 |
| L-menthol | 2.51 | 2.44 |
| Methanol (% of wet film) | 77.2 | 77.2 |
| Water (% of wet film) | 22.8 | 22.8 |

Note: All ingredient amount is in % w/w to dry film except organic solvents and water.

TABLE 7

Salvinorin A Film Formulations

| Ingredient | Formulation 9 | Formulation 10 | Formulation 11 | Formulation 12 |
|---|---|---|---|---|
| | Composition (% w/w) | | | |
| Salvinorin A | 2.98 | 3.04 | 3.01 | 3.06 |
| Copovidone | 34.12 | 34.85 | 34.52 | 35.04 |
| Hydroxypropyl Cellulose | 34.12 | 34.85 | 34.52 | 35.04 |
| Gelucire 44/14 | 5.11 | - | 5.17 | 5.25 |
| Linoleoyl Polyoxyl-6 glycerides | 2.10 | - | 2.13 | 2.16 |
| Polyethylene glycol (300) | 4.67 | 4.77 | 6.21 | 6.30 |
| Sucralose | 1.46 | 1.49 | 1.48 | 1.50 |
| Maltitol | 4.38 | 2.98 | - | - |
| L-menthol | 1.56 | 1.60 | 1.58 | 1.60 |
| Benzyl Alcohol | 9.49 | 9.70 | 6.95 | 7.05 |
| Vitamin E TPGS | - | 6.71 | - | - |
| Chlorophyll | - | - | 1.48 | - |
| Camphor | - | - | 1.48 | - |
| Borneol | - | - | 1.48 | - |
| Chitosan Nanoparticle Suspension | - | - | - | 5.83 |
| MEK (% of wet film) | 66.67 | 66.67 | 66.67 | 66.67 |
| Ethanol (% of wet film) | 33.33 | 33.33 | 33.33 | 33.33 |

Note: All ingredient amount is in % w/w to dry film except organic solvents.

### Example 1.4 Solubility of Salvinorin A and Salvinorin A Loaded Films

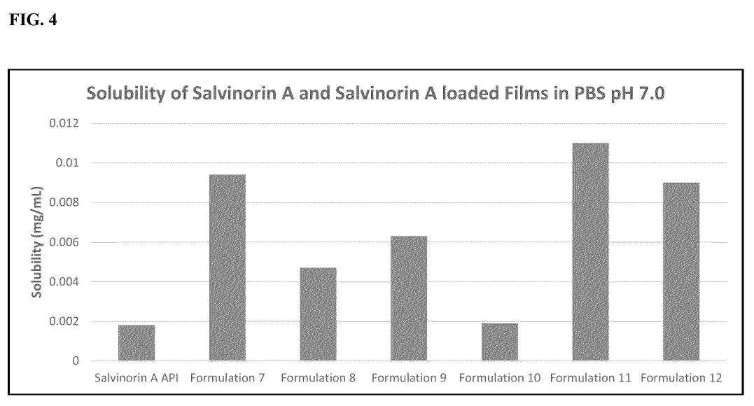

The solubility of Salvinorin A API and Salvinorin A loaded buccal films are evaluated by mixing 4 mg equivalent sample of API powder or film in 10 mL of phosphate buffer saline pH 7 (10 mM PBS, pH 7) for 3 hours. After 3 hours samples were filtered through 0.45/0.22 micron filter and tested for content by HPLC analysis. Results of the solubility study is presented below in Table 8 and depicted in FIG. 4.

TABLE 8

Solubility of Salvinorin A Films

| Lot # | Sal A API | Form 7 | Form 8 | Form 9 | Form 10 | Form 11 | Form 12 |
|---|---|---|---|---|---|---|---|
| Solubility (mg/mL) | 0.0018 | 0.0094 | 0.0047 | 0.0063 | 0.0019 | 0.011 | 0.0090 |

### Example 1.5 Dissolution of Salvinorin A Loaded Films

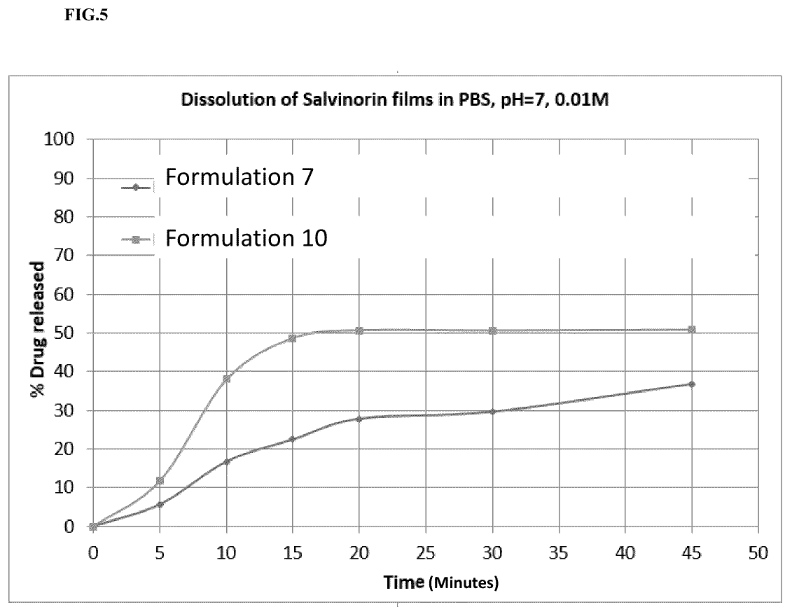

As shown in Table 8, Salvinorin A API powder exhibited very low solubility in 10 mM PBS, pH 7.0. To analyze release characteristics of Salvinorin A from the film formulations dissolution of films was evaluated. Dissolution of formulation equivalent to Formulations 7 (circles) and 10 (squares) were carried out in 10 mM PBS, pH 7.0 using a paddle over disk apparatus and results are depicted in FIG. 5. As FIG. 5 illustrates, both the films demonstrated drug release from the film formulation despite low solubility of Salvinorin A API powder in aqueous dissolution media.

All the above data demonstrates that film-based formulations comprising Salvinorin A in accordance with the disclosure resulted in increased Salvinorin A solubility as compared to Salvinorin A API powder in saline pH 7 which simulates the pH of the buccal cavity. Based on the in vitro solubility and dissolution data, it is believed that Salvinorin A formulated as a buccal film can provide adequate Salvinorin A solubility, making it available for absorption through buccal mucosa and into the bloodstream.

### Example 2: Salvinorin A Injectable Formulations

### Example 2.1. Solubility Assessment for Injectable Formulations

Development of liquid formulations of Salvinorin A (e.g., injectable) is challenging due to its known poor solubility in different solvents and vehicles. A series of solubility studies were carried out to evaluate solvent systems that may be suitable for various injectable formulations (i.e., subcutaneous, interperitoneal, intravenous, etc.) comprising Salvinorin A or a derivative of Salvinorin A. In addition to the solvents and vehicles described below, various techniques can be utilized to achieve complete solubilization of Salvinorin A, including but not limited to microemulsion, nanoemulsions, polymeric nanoparticles, polymeric micelles, nanostructured lipid carriers, liposomes, transformers and the like.

In Table 9 below the solubility of Salvinorin A in polar aprotic solvents was evaluated by increasing the amount of Salvinorin A in the candidate solvents.

TABLE 9

Salvinorin A solubility in polar aprotic solvents

| Solvent | Conc (mg/ml) | Solubility (Y= soluble, N= not soluble completely) |
|---|---|---|
| DMA | 100 | Y |
| DMA | 200 | N |
| DMSO | 100 | Y |
| DMSO | 200 | N |
| NMP | 20 | Y |
| NMP | 100 | Y |
| NMP | 200 | Y |

DMA = Dimethylacetamide; DMSO = Dimethylsulfoxide; NMP = N-Methylpyrrolidone

Following the above, aqueous and organic vehicles were combined with aprotic solvents and screened. The range of target Salvinorin A concentration was from 5 mg/ml to 20 mg/ml. The results are presented in Table 10 below.

TABLE 10

Salvinorin A solubility in polar aprotic solvents + vehicles

| Form. | Vehicle | Conc. (mg/ml) | Solubility (Y= soluble; N= not soluble completely) |
|---|---|---|---|
| 1 | Corn oil | 5 | N |
| 2 | NMP : PEG 400 : 1% Tween 80, 0.5% HPMC (aqueous) (1:1:1 v/v/v) | 6 | N |
| 3 | NMP : EtOH : PEG 400 : 1% Tween 80, 0.5% HPMC (aqueous) (1:1:2:2 v/v/v/v) | 10 | N |
| 4 | DMSO : PEG400 (1:3 v/v) | 25 | Y |
| 5 | DMSO : PEG400 : 1% Tween 80, 0.5% HPMC (aqueous) (1:3:1 v/v/v) | 5 | N |
| 6 | DMSO : EtOH : PEG 400 : Labrasol (1:1:4:4 v/v/v/v) | 5 | Y |
| 7 | DMSO : EtOH : PEG 400 : Labrasol (1:1:4:4 v/v/v/v) | 10 | Y, precipitate over night |
| 8 | NMP : PEG 400 : Labrasol (1:4.5:4.5 v/v/v) | 5 | Y |
| 9 | NMP : PEG 400 : Labrasol (1:4.5:4.5 v/v/v) | 10 | Y |
| 10 | NMP : PEG 400 : Labrasol (1:4.5:4.5 v/v/v) | 15 | Y, precipitate over night |
| 11 | NMP : PEG 400 : Labrasol (1:4.5:4.5 v/v/v) | 20 | Y, precipitate over night |
| 12 | NMP : PG (1:9 v/v) | 5 | N |
| 13 | NMP : PEG400 : PG (1:4.5:4.5 v/v/v) | 5 | Y |
| 14 | NMP : PEG400 : PG (1:4.5:4.5 v/v/v) | 10 | Y, precipitate over night |
| 15 | NMP : PEG400 : PG (1:4.5:4.5 v/v/v) | 15 | N |
| 16 | NMP : Labrasol (1:9 v/v) | 5 | Y |
| 17 | NMP : Labrasol (1:9 v/v) | 10 | Y |
| 18 | NMP : Labrasol (1:9 v/v) | 20 | N |

HPMC = Hydroxypropyl methyl cellulose; EtOH = Ethanol; PG = Propylene glycol; Labrasol® = surfactant comprising caprylocaproyl macrogolglycerides (polyoxyglycerides) primarily PEG-8 (MW 400) mono- and diesters of caprylic (C8) and capric (C10) acids, with a small fraction of mono-, di-, and triglycerides.

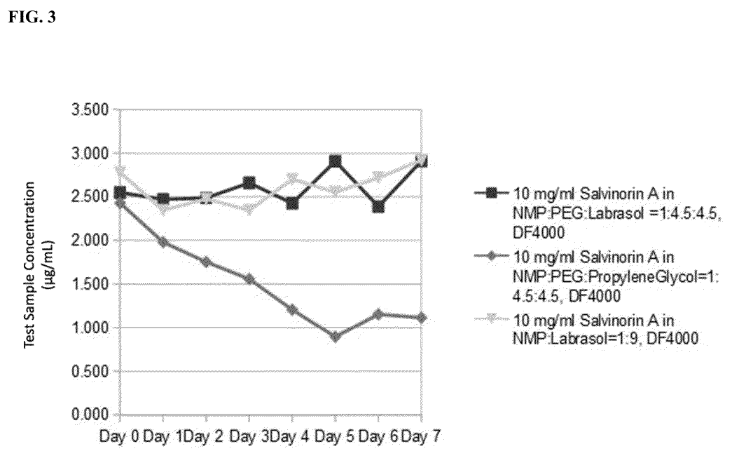

Three of the formulations were selected and submitted for stability assessment. A calibration curve was produced by injection of Salvinorin A standards with concentrations of 0.1 µg/ml, 0.25 µg/ml, 0.5 µg/ml, 1 µg/ml, 4 µg/ml. The standard solutions were prepared by diluting 1 mg/ml of Salvinorin A in acetonitrile (ACN) with 25% ACN/water. Samples for stability assessment were prepared from formulations with a dilution factor (DF) of 4000 (20 µl of formulation mixture was mixed with 480 µl of DMSO, and vortexed). 12.5 µl of DMSO solution of formulation was then mixed with 487.5 µl of 25% ACN/Water, desired concentration 2.5 µg/ml). The samples were submitted to HPLC-MS at one time point at about 24 hour intervals during a 7-day period. The results are presented in FIG. 3. All three solvent combinations showed adequate stability. The NMP : PEG 400 : Propylene glycol formulation showed a concentration decline over time due to observed precipitation.

Additional vehicles, solvents, and combinations thereof were evaluated for Salvinorin A solubility. The results are presented in Table 11 below. For trials F1 to F41, 50 mg of Salvinorin A was mixed in 5 mL of vehicle(s) and stirred overnight under ambient conditions and protected from light. For trials F42 to F49, 150 mg of Salvinorin A was mixed with 5 mL of vehicle(s) and stirred overnight under ambient condition and protected from light.

Following the overnight stirring, samples were centrifuged, and supernatant was collected and analyzed using HPLC to estimate solubility of Salvinorin A.

TABLE 11

Salvinorin A solubility in vehicles

| Trial | Vehicle | Solubility (mg/mL) |
|---|---|---|
| F1 | PEG300 | 3.177 |
| F2 | MCT | 0.3577 |
| F3 | Castor Oil | 1.1515 |
| F4 | Sesame Oil | 0.191 |
| F5 | Benzyl alcohol | 9.4395 |
| F6 | Benzyl benzoate | 6.5679 |
| F7 | 10% w/v Tween 80 in WFI | 0.0179 |
| F8 | 50% w/v Tween 80 in WFI | 0.0949 |
| F9 | 10% w/v Kolliphor ELP in WFI | 0.0172 |
| F10 | 50% w/v Kolliphor ELP in WFI | 0.9505 |
| F11 | 10% w/v Tween 20 in WFI | 0.0162 |
| F12 | 10% w/v Kolliphor HS 15 in WFI | 0.0131 |
| F13 | 10% w/v Poloxamer 188 in WFI | 0.0013 |
| F14 | 30% w/v SBEbCD in WFI | 0.0825 |
| F15 | 30% w/v HPbCD in WFI | 1.4695 |
| F16 | 40% w/v HP-β-CD in WFI | 0.3169 |
| F17 | 50% v/v Benzyl benzoate + 50% v/v PEG 300 | 4.7571 |
| F18 | 50% v/v Benzyl benzoate + 50% v/v Castor oil | vehicles not miscible |
| F19 | 9% v/v Benzyl alcohol + 41% v/v Benzyl benzoate + 50% v/v PEG 300 | 4.9173 |
| F20 | 50% v/v Benzyl benzoate + 30% v/v Propylene Glycol (PG) + 20% v/v Ethanol | vehicles not miscible |
| F21 | 41% v/v Benzyl benzoate + 9% v/v Benzyl alcohol + 30% v/v PEG 300 + 20% v/v Ethanol | 6.1356 |
| F22 | 10% v/v NMP + 90% v/v PEG 300 | 3.9532 |
| F23 | 10% v/v NMP + 50% v/v Benzyl benzoate + 40% v/v PEG 300 | 6.3081 |
| F24 | 10% v/v NMP + 50% v/v Benzyl benzoate + 40% v/v PG | vehicles not miscible |
| F25 | 10% v/v NMP + 50% v/v Benzyl benzoate + 20% v/v PEG 300+ 20% Ethanol | 7.0824 |
| F26 | 10% v/v NMP + 30% v/v Benzyl benzoate + 40% v/v PEG 300+ 20% Ethanol | 6.0618 |
| F27 | 100% PG | 0.1304 |
| F28 | 100% Ethanol | 0.5571 |
| F29 | 100% NMP | 9.0152 |
| F30 | 20% v/v NMP + 50% v/v Benzyl benzoate + 30% v/v PEG 300 | 9.6848 |
| F31 | 30% v/v NMP + 50% v/v Benzyl benzoate + 20% v/v PEG 300 | 9.5447 |
| F32 | 30% v/v NMP + 30% v/v Benzyl benzoate + 40% v/v PEG 300 | 9.0466 |
| F33 | 30% v/v NMP + 70% v/v PEG 300 | 8.7062 |
| F34 | 30% v/v NMP + 50% v/v PEG 300 + 20% v/v WFI | 1.259 |
| F35 | 30% v/v NMP + 30% v/v PEG 300 + 40% v/v WFI | 0.2624 |
| F36 | 5% w/v Sodium Deoxycholate in WFI | 0.0356 |
| F37 | 10% Lecithin E80 w/w + 90% w/w Sesame Oil | vehicles not miscible |
| F38 | 10% Lecithin S100 w/w + 90% w/w Sesame Oil | vehicles not miscible |
| F39 | 30% HPβCD, 20% PEG 300, 9% Benzyl Alcohol, 41% Water | 0.0862 |
| F40 | 30% HPβCD, 20% PEG 300, 10% NMP, 40% Water | 0.5912 |
| F41 | 30% HPβCD, 20% PEG 300, 50% Water | 0.2852 |
| F42 | 30% v/v NMP + 70% v/v PEG 300 (API added in mixed vehicle) | 11.651 |
| F43 | 20% v/v NMP + 80% v/v PEG 300 (API added in mixed vehicle) | 7.571 |
| F44 | 30% v/v NMP + 70% v/v PEG 300 (API added in NMP and then add PEG300) | 21.529 |
| F45 | 20% v/v NMP + 80% v/v PEG 300 (API added in NMP and then add PEG300) | 9.007 |
| F46 | 30% v/v NMP + 70% v/v PEG 300 (API added in mixed vehicle) | 12.858 |
| F47 | 20% v/v NMP + 80% v/v PEG 300 (API added in mixed vehicle) | 8.502 |
| F48 | 30% v/v NMP + 70% v/v PEG 300 (API added in NMP and then add PEG300) | 14.95 |
| F49 | 20% v/v NMP + 80% v/v PEG 300 (API added in NMP and then add PEG300) | 9.462 |

WFI = Water for injection, HPβCD = Hydroxypropyl β Cyclodextrin,

All the solubility data demonstrates that the solubility of Salvinorin A can be manipulated through adjustment of vehicles (solvents, co-solvents), which allows selection of particular vehicle/solvent systems (i.e., solvent and/or co-solvent and/or mixtures) for the development of Salvinorin A formulations adaptable for subcutaneous injection.

### Example 2.2. Initial Injectable Formulations

A series of Salvinorin A liquid formulations are prepared based on the general ranges in Table 12 below, and are evaluated for various parameters as an injectable (e.g., subcutaneous).

TABLE 12

Salvinorin A injectable formulations

| | | |
|---|---|---|
| Ingredient | Formulation 1 | Formulation 2 |
| Salvinorin A | 0.01-10% | 0.01-10% |
| Complexing agent (e.g. a cyclodextrin or its derivative) | - | 5-50% |
| S tabilizer/antioxidant | 0-5% | 0-5% |
| Buffering agent(s) | 0-5% | 0-5% |
| pH modifying agent(s) (e.g. NaOH and or HC1) | 0-5% | 0-5% |
| Vehicle* | to 100% | to 100% |

* Mixture of solvent, cosolvents and surfactant

### Example 2.2. Subcutaneous Injectable Formulations

An initial series of formulations for subcutaneous injection are prepared according to Table 13 and evaluated for performance characteristics.

TABLE 13

Salvinorin A Subcutaneous Injection Formulations

| Ingredient (% w/v) | Form 1 (sc) | Form 2 (sc) | Form 3 (sc) | Form 4 (sc) |
|---|---|---|---|---|
| Salvinorin A | 0.01-1 | 0.5 | 0.5 | 0.5 |
| N-methyl pyrrolidone (NMP) | 0-100 | 20 | 30 | 10 |
| Polyethylene glycol (PEG300) | 0-100 | 79.5 | 69.5 | 89.0 |
| S tabilizer/antioxidant | 0-5 | - | - | - |
| Precipitation Inhibitors (e.g., PVP K-90) | 0-5 | - | - | 0.5 |

* Non-limiting examples of solvents /co-solvents include the following combinations:
80% NMP / 20% PEG 300; 30% NMP / 70% PEG 300; 10% NMP / 90% PEG 400; 90% NMP / 10% PEG 400; 30% NMP / 70% PEG 400.

### Example 2.3. Stability of Subcutaneous Injectable Formulations

A representative formulation for subcutaneous injection is prepared and assessed for its ability to stabilize Salvinorin A. Briefly, an amount of Salvinorin A is weighed and mixed with N-Methylpyrrolidone (NMP) and stirred till it dissolved. PEG 300 then added to the NMP solution with continuous mixing and stirred till clear solution is achieved. The volume of composition was then made up with further addition of PEG 300. The resulting solution is filtered and filled in glass vials (clear or amber) with or without nitrogen purging and closed with rubber stopper and flipoff seals.

TABLE 14

Formulations for Stability Testing

| | |
|---|---|
| Ingredient | Formulation (%w/v) |
| Salvinorin A | 0.05 |
| N-Methylpyrrolidone (NMP) | 30 |
| Polyethylene Glycol 300 (PEG300) | Q.S to 1 mL |

One set of formulation was prepared using without nitrogen purging. Another set was prepared by purging head space with Nitrogen, to achieve head space oxygen < 5%.

Samples were evaluated for stability at 2-8° C., 25° C. / 60% RH (upright orientation) and at 60° C. (upright and inverted orientation). The samples were analyzed for appearance, assay and impurity / degradation level utilizing HPLC method, with the results summarized in the following Tables.

TABLE 15

Stability of Salvinorin A Solution 5 mg/mL (no N2 purge), Various Conditions
Stability Condition: 2-8° C.
Orientation: Upright

| | | | | | |
|---|---|---|---|---|---|
| Time | Initial | 1 Week | 2 Week | 3 Week | 4 Week |
| Appearance | Clear, colorless solution free of visible particulates | Clear, colorless solution free of visible particulates | Clear, colorless solution free of visible particulates | Clear, colorless solution free of visible particulates | Clear, colorless solution free of visible particulates |
| Assay (% LC) | 97.7% | 96.5% | 98.8% | 97.9% | 98.0% |
| Impurity (#peaks & % area) | 7 peaks Total: 1.5% | 6 peaks Total: 2.2% | 5 peaks Total: 1.6% | 6 peaks Total: 1.8% | 6 peaks Total: 1.4% |

Stability Condition: 25±°C / 60±5% RH
Orientation: Upright

| | | | | | |
|---|---|---|---|---|---|
| Time | Initial | 1 Week | 2 Week | 3 Week | 4 Week |
| Appearance | Clear, colorless solution free of visible particulates | Clear, colorless solution free of visible particulates | Clear, colorless solution free of visible particulates | Clear, colorless solution free of visible particulates | Clear, colorless solution free of visible particulates |
| Assay (% LC) | 97.7% | 95.7% | 93.1% | 90.7% | 94.2% |
| Impurity (#peaks & % area) | 7 peaks Total: 1.5% | 5 peaks Total: 0.5% | 4 peaks Total: 1.0% | 8 peaks Total: 0.9% | 17 peaks Total: 5.1% |

Stability Condition: 60° C.
Orientation: Inverted

| | | |
|---|---|---|
| Time | Initial | 4 Week |
| Appearance | Clear, colorless solution free of visible particulates | Clear, colorless solution free of visible particulates |
| Assay (% LC) | 97.7% | 94.7% |
| Impurity (# peaks & % area) | 7 peaks Total: 1.5% | 17 peaks Total: 4.9% |

TABLE 16

Stability of Salvinorin A Solution 5 mg/mL (N2 purge), Various Conditions
Stability Condition: 25±2° C./60±5% RH
Orientation: Upright

| | | | | | |
|---|---|---|---|---|---|
| Time | Initial | 1 Week | 2 Week | 3 Week | 4 Week |
| Appearance | Clear, colorless solution free of visible particulates | Clear, colorless solution free of visible particulates | Clear, colorless solution free of visible particulates | Clear, colorless solution free of visible particulates | Clear, colorless solution free of visible particulates |
| Assay (% LC) | 99.2% | 98.6% | 99.0% | 100.2% | 98.4% |
| Impurity (# peaks & % area) | 7 peaks Total: 1.2% | 6 peaks Total: 0.9% | 5 peaks Total: 0.9% | 5 peaks Total: 0.8% | 8 peaks Total: 0.9% |
| | visible particulates | visible particulates | visible particulates | visible particulates | visible particulates |
| Appearance | Clear, colorless solution free of visible particulates | Clear, colorless solution free of visible particulates | | | |
| Assay (% LC) | 99.2% | 98.4% | | | |
| Impurity (# peaks & % area) | 7 peaks Total: 1.2% | 11 peaks Total: 1.2% | | | |

Stability Condition: 60° C.
Orientation: Upright

| | | | | | |
|---|---|---|---|---|---|
| Time | Initial | 1 Week | 2 Week | 3 Week | 4 Week |
| Appearance | Clear, colorless solution free | Clear, colorless solution free of | Clear, colorless solution free of | Clear, colorless solution free of | Clear, colorless solution free of |
| | of visible particulates | of visible particulates | of visible particulates | of visible particulates | of visible particulates |
| Assay (% LC) | 99.2% | 98.4% | 98.2% | 98.0% | 97.7% |
| Impurity (# peaks & % area) | 7 peaks Total: 1.2% | 5 peaks Total: 0.4% | 3 peaks Total: 0.4% | 4 peaks Total: 0.4% | 12 peaks Total: 1.3% |

Stability Condition: 60° C.
Orientation: Inverted

| | | |
|---|---|---|
| Time | Initial | 4 Week |
| Appearance | Clear, colorless solution free of visible particulates | Clear, colorless solution free of visible particulates |
| Assay (% LC) | 99.2% | 98.4% |
| Impurity (# peaks & % area) | 7 peaks Total: 1.2% | 11 peaks Total: 1.2% |

Further, the formulations can be screened for possible post injection events like precipitation upon injection which in turn might cause local irritation and may delay the absorption in to blood by dilution with suitable aqueous or simulated media. SCISSOR (Subcutaneous Injection Site Simulator) is an in vitro technique that utilizes is an instrument designed to simulate the stress conditions and environmental transitions that a biopharmaceutical, peptide or small molecule drug experiences when injected into a subcutaneous environment. These include chemical stresses (such as buffer composition, pH change and loss of excipients) and physical stresses (such as temperature and pressure changes).

Stability of samples with and without nitrogen blanket was evaluated at 2-8° C., 25° C. /60% RH (upright orientation) and at 60° C. (upright and inverted orientation). Stability of samples were analyzed for appearance, assay and impurity / degradation level utilizing HPLC method. Stability results demonstrated acceptable stability for all the samples under the studied storage conditions, for up to 4 weeks.

### Example 3: Pharmacokinetics of Salvinorin A Injectable Formulations

A series of experiments were conducted in order to evaluate the pharmacokinetics of injectable Salvinorin A compositions in rats. In the first experiment Salvinorin A was formulated at two different dose concentrations (5 mg/mL and 20 mg/mL Salvinorin A in 1-methyl-2-pyrrolidine, polyethylene glycol 400 at a 1:9 v/v ratio) and administered via injection (subcutaneous) to two groups of male Sprague-Dawley rats. The pharmacokinetic data is summarized in Table 17.

TABLE 17

Pharmacokinetics of Subcutaneous Salvinorin A

| Group | Dose SalA (mg/kg) | Rat | Tₘₐₓ (h) | T_{½} (h) | Cₘₐₓ (ng/mL) | AUCₗₐₛₜ (h*ng/mL) | AUC₀-₀₀, (h*ng/mL) |
|---|---|---|---|---|---|---|---|
| 1 | 25 | 0 | 0.50 | 1.00 | 209 | 362 | 389 |
| | | 1 | 0.50 | 1.58 | 213 | 405 | 422 |
| | | 2 | 0.50 | 1.96 | 224 | 515 | 560 |
| | | Mean | 0.50 | 1.5 | 215.3 | 427.3 | 457.0 |
| | | SD | 0.0 | 0.5 | 7.8 | 78.9 | 90.7 |
| 2 | 100 | 3 | 1.00 | 0.87 | 187 | 348 | 365 |
| | | 4 | 0.50 | 1.91 | 208 | 461 | 498 |
| | | 5 | 0.50 | 1.73 | 155 | 376 | 395 |
| | | Mean | 0.7 | 1.5 | 183.3 | 395.0 | 419.3 |
| | | SD | 0.3 | 0.6 | 26.7 | 58.8 | 69.8 |

In another experiment Salvinorin A was formulated at three different dose concentrations (1 mg/mL, 5 mg/mL, and 10 mg/mL Salvinorin A in 1-methyl-2-pyrrolidine, polyethylene glycol 400 at a 1:9 v/v ratio) and administered via injection (subcutaneous) to three groups of Sprague-Dawley rats (each group including female and male rats). The formulation containing 10 mg/mL Salvinorin A was administered as two injections at separate dosage sites (total dose of 100 mg/kg). The same groups of rats were administered a second dose of the injectable composition 7 days after the first dosing. The pharmacokinetic data is summarized in Tables 18-19.

TABLE 18

Pharmacokinetics of Subcutaneous Salvinorin A (Day 1)

| Group | Dose SalA (mg/kg) | Sex | Tₘₐₓ (h) | T_{½} (h) | Cₘₐₓ (ng/mL) | Tₗₐₛₜ (h) | AUCₗₐₛₜ (h*ng/mL) | AUC₀₋₀₀ (h*ng/mL) |
|---|---|---|---|---|---|---|---|---|
| 10 | 5 | M | 2 | NC¹ | 58.4 | 4 | 148 | NC |
| | | F | 4 | NC | 75.8 | 4 | 233 | NC |
| | | Mean | 3 | NA² | 67.1 | | 191 | NA |
| 11 | 25 | M | 1 | NC | 135 | 4 | 361 | NC |
| | | F | 0.5 | NR³ | 250 | 4 | 389 | NR |
| | | Mean | 0.75 | NA | 192 | | 375 | NA |
| 12 | 100 | M | 0.5 | 4.78 | 321 | 24 | 1210 | 1240 |
| | | F | 1 | 4.80 | 419 | 24 | 1870 | 1920 |
| | | Mean | 0.075 | 4.79 | 370 | | 1540 | 1580 |

TABLE 19

Pharmacokinetics of Subcutaneous Salvinorin A (Day 8)

| Group | Dose SalA (mg/kg) | Sex | Tmax (h) | (h) | Cmax (ng/mL) | Tₗₐₛₜ (h) | AUCₗₐₛₜ (h*ng/mL) | AUC₀₋₀₀ (h*ng/mL) |
|---|---|---|---|---|---|---|---|---|
| 10 | 5 | M | 2 | NC | 70.6 | 4 | 188 | NC |
| | | F | 2 | NC | 78.3 | 4 | 211 | NC |
| | | Mean | 2 | NA | 74.5 | | 200 | NA |
| 11 | 25 | M | 1 | 4.92 | 185 | 24 | 500 | 512 |
| | | F | 0.5 | 4.16 | 162 | 24 | 946 | 958 |
| | | Mean | 0.75 | 4.54 | 174 | | 723 | 735 |
| 12 | 100 | M | 0.5 | 5.20 | 368 | 24 | 1280 | 1320 |
| | | F | 0.5 | 4.80 | 315 | 24 | 1470 | 1520 |
| | | Mean | 0.05 | 5.00 | 341 | | 1380 | 1420 |

¹NC = not calculated (insufficient data points in the elimination phase)
²NA = not applicable (fewer than 2 values for mean)
³NR = not reported (extensive extrapolation (>30%) for the elimination phase)

Following subcutaneous dosing in the rat in two separate studies, the time to peak plasma concentration (Tₘₐₓ) ranged from 0.5 hours following dosing at 25 and 100 mg/kg to 2-4 hours following dosing at 5 mg/kg. This data indicates a role for dose level, dose concentration and route of administration in modulating the rate of rise to peak concentration as indicated by the range of acheivable Tₘₐₓ values. This interaction between dosing route and dose level/concentration was observed after a single dose and a second dose administered 7 days later.

1. A composition comprising:
(i) Salvinorin A or a derivative of Salvinorin A;
(ii) a vehicle adapted for subcutaneous injection comprising
(a) a solvent including a pyrrolidone or an oil;
(b) a co-solvent including polyethylene glycol; and
(c) a surfactant including a fatty acid ester.

2. The composition according to claim 1, wherein the pyrrolidone comprises N-methyl pyrrolidone (NMP), the polyethylene glycol comprises PEG300 or PEG400, and the fatty acid ester comprises caprylocaproyl macrogolglycerides.

3. The composition according to claim 1, wherein the vehicle further comprises a complexing agent comprising a cyclodextrin.

4. The composition according to claim 3, wherein the cyclodextrin comprises 2-hydroxypropyl-beta-cyclodextrin.

5. The composition according to claim 1, wherein the vehicle further comprises a stabilizer, surfactant, precipitation inhibitor, preservative, and/or an antioxidant.

6. The composition according to claim 5, wherein the amount of Salvinorin A in the composition comprises about 0.01% to about 10.0 %.

7. A buccal film comprising
(i) Salvinorin A or a derivative of Salvinorin A; and
(ii) a film matrix adapted for buccal administration comprising:
(a) a solvent comprising N-methylpyrrolidone, methanol, methyethylketone, acetone, or a combination thereof:
(b) a co-solvent;
(c) a surfactant; and
(d) a polymer comprising copovidone, hydroxypropyl cellulose, povidone, or a combination thereof;
wherein said Salvinorin A or Salvinorin A derivative is dispersed within said film matrix.

8. The buccal film of claim 7, wherein the surfactant comprises Lauroyl Polyoxyl-32 glycerides, D-α-tocopheryl polyethylene glycol succinate or a combination thereof.

9. The buccal film of claim 7, wherein the co-solvent comprises propylene glycol, benzyl alcohol or a combination thereof.

10. The buccal film of claim 7, wherein the film matrix further comprises a permeability enhancer and/or an oil.

11. The buccal film of claim 7, comprising about 0.01% to about 10.0% (w/w) of Salvinorin A or Salvinorin A derivative.

12. A composition comprising a pharmaceutically effective amount of Salvinorin A or a derivative of Salvinorin A, wherein said composition is capable, following administration to a human subject, of providing a human blood plasma Tₘₐₓ of Salvinorin A or Salvinorin A derivative ranging from about 10 minutes to about 240 minutes.

13. The composition according to claim 12, which is capable, after Tₘₐₓ is achieved, of maintaining the concentration of Salvinorin A or derivative of Salvinorin A at about 50% or more of Cₘₐₓ for between about 10 minutes to about 90 minutes.

14. The composition according to claim 12, which is capable, after Tₘₐₓ is achieved, of maintaining the concentration of Salvinorin A or a derivative of Salvinorin A at about 80% or more of Cₘₐₓ for between about 10 minutes to about 45 minutes.

15. The composition according to claim 12, which is capable, following administration to a human subject, of maintaining therapeutically effective blood levels of Salvinorin A or Salvinorin A derivative for at least about 20 minutes.

16. The composition according to claim 15, capable of maintaining therapeutically effective blood levels of Salvinorin A or Salvinorin A derivative for between about 20 minutes to about 240 minutes.

17. The composition according to claim 15, capable of maintaining therapeutically effective blood levels of Salvinorin A or Salvinorin A derivative for between about 20 minutes to about 180 minutes.

18. The composition according to claim 15, capable of maintaining therapeutically effective blood levels of Salvinorin A or Salvinorin A derivative for between about 20 minutes to about 60 minutes.

19. The composition according to claim 15, capable of maintaining therapeutically effective blood levels of Salvinorin A or Salvinorin A derivative for between about 40 minutes to about 60 minutes.

20. The composition according to claim 12 comprising a pharmaceutically effective amount of Salvinorin A or a derivative of Salvinorin A, which is capable, following administration to a human subject, of releasing at least 50% of the Salvinorin A or the derivative of Salvinorin A from the composition within about 10 minutes to about 60 minutes.

21. The composition according to claim 12, capable of providing a human blood plasma T½ of about 15 minutes to about 240 minutes.

22. The composition according to claim 21, capable of providing a human blood plasma T½ of about 15 minutes to about 90 minutes.

23. The composition according to claim 12, wherein administration comprises buccal delivery or subcutaneous injection.

24. The composition according to claim 12, wherein the pharmaceutically effective amount in the composition is effective to treat a neurological disease or condition.

25. The composition according to claim 12, comprising Salvinorin A.

26. The composition according to claim 12, wherein the pharmaceutically effective amount of Salvinorin A or Salvinorin A derivative is from about 100 µg to about 10,000 µg.

27. The composition according to claim 12, comprising from about 1.5 µg/kg to about 16.5 µg/kg of Salvinorin A or Salvinorin A derivative.

28. (canceled)

29. The composition according to claim 12, wherein the Salvinorin A or a Salvinorin A derivative is adapted for buccal administration.

30. The composition according to claim 12, wherein the Salvinorin A or a Salvinorin A derivative is adapted for subcutaneous injection.

31. The composition according to claim 29 comprising a buccal formulation comprising a tablet, rapidly dissolving tablet, wafer, film, strip or patch, oro-dispersible tablet, oral gel, medicated lollipop, spray, or drops.

32. The composition according to claims 30 comprising a formulation adapted for subcutaneous injection, comprising a ready-to-use solution or a lyophilized composition.

33. A method of treating a neurological disease or condition comprising administration by buccal delivery or by subcutaneous injection to a human subject of an effective amount of Salvinorin A or a derivative of Salvinorin A in a composition according to claim 12.

34. The method of treatment according to claim 33, wherein the neurological disease or condition is a neuropsychiatric disease or disorder comprising depression, treatment-resistant depression (TRD), anxiety, bipolar disorder, post-traumatic stress disorder (PTSD), abnormalities of mood or emotion, dysthymia, schizoaffective disorder, schizophrenia and other psychotic disorders, panic disorder, traumatic stress disorders, phobic disorders, and personality disorders with abnormal mood, borderline personality disorder, schizoid and schizotypal disorders, suicide ideation, or rumination/unproductive repetitive thoughts that negatively impact behavior, mood, and/or ability to focus.

35. The method of treatment according to claim 33, wherein the neurological disease or condition is addiction comprising substance use disorder, addiction to nicotine, addiction to alcohol, addiction to cocaine, addiction to opioids, addiction to amphetamine, addiction to methamphetamine, addiction to heroin, addiction to morphine, addiction to phencyclidine, addiction to 3,4-methylenedioxy-methamphetamine, or other addictive substances.

36. The method of treatment according to claim 33, wherein the neurological disease or condition is an addictive behavior comprising addiction to eating, addiction to gambling, addiction to sex, addiction to pornography, addiction to videogames, addiction to work, addiction to exercise, addiction to spiritual obsession, self-harm, addiction to travel or addiction to shopping.

37. The method of treatment according to claim 33, wherein the neurological disease or condition is pain, migraine, headache, orofacial pain, or chronic pain.

38. (canceled)

39. The method of treatment according to claim 33, wherein Salvinorin A or a derivative of Salvinorin A is co-administered with one or more additional therapeutic agents.

40. The method of treatment according to claim 39, wherein the one or more additional therapeutic agents are administered as one or more separate compositions.

41. A method of treating depression, treatment-resistant depression (TRD), pain, or an addiction, comprising administering to a human subject in need of treatment, an effective amount of a buccal or injectable composition comprising Salvinorin A or a derivative of Salvinorin A, according to claim 12, in conjunction with one or more of psychotherapy, talk therapy, cognitive behavioral therapy, exposure therapy, biofeedback therapy, systematic desensitization, mindfulness, dialectical behavior therapy, interpersonal therapy, eye movement desensitization and reprocessing, social rhythm therapy, acceptance and commitment therapy, family-focused therapy, psychodynamic therapy, light therapy, computer therapy (including digital cognitive behavioral therapy), cognitive remediation, exercise, TMS, or other therapies.

42. The method of treatment according to claim 33 comprising Salvinorin A.

43. The composition according to claim 12 comprising Salvinorin A, capable, following buccal administration to a human subject, of achieving a Salvinorin A human blood plasma Tₘₐₓ of from about 10 minutes to about 240 minutes, and capable, after Tₘₐₓ is achieved, of maintaining the concentration of Salvinorin A for about 80% or more of Cₘₐₓ for between about 10 minutes to about 45 minutes.

44. The composition according to claim 12 comprising Salvinorin A, capable, following administration by subcutaneous injection to a human subject, of achieving a Salvinorin A human blood plasma Tₘₐₓ of from about 10 minutes to about 240 minutes, and capable, after Tₘₐₓ is achieved, of maintaining the concentration of Salvinorin A at about 80% or more of Cₘₐₓ for between about 10 minutes to about 45 minutes.
